(19) United States
(12) Patent Application Publication
(10) Pub. No.: US 2025/0257048 A1
(43) Pub. Date: Aug. 14, 2025

(54) METHODS OF PREPARING AN ISOINDOLINONE DERIVATIVE AND CRYSTAL FORMS THEREOF

(71) Applicant: Monte Rosa Therapeutics, Inc., Boston, MA (US)

(72) Inventors: Simone Tortoioli, Basel (CH); Bernhard Fasching, Basel (CH)

(21) Appl. No.: 19/018,468

(22) Filed: Jan. 13, 2025

Related U.S. Application Data
(63) Continuation of application No. PCT/US2023/027760, filed on Jul. 14, 2023.
(60) Provisional application No. 63/389,155, filed on Jul. 14, 2022.

(51) Int. Cl. C07D 401/04; A61K 31/454

(52) U.S. Cl. CPC C07D 401/04; A61K 31/454

(57) ABSTRACT

The present disclosure provides, in certain embodiments, crystalline forms of and a process for preparing a compound having the structure:

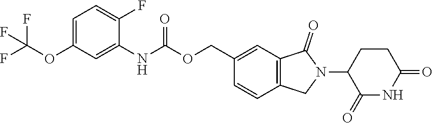 [SMILES: O=C1CCC(N2Cc3ccc(COC(=O)Nc4cc(OC(F)(F)F)ccc4F)cc3C2=O)C(=O)N1]

which is contemplated as a molecular glue that binds cereblon and mediate the degradation of a protein.

### CROSS-REFERENCE TO RELATED APPLICATIONS

This application is a continuation of International Patent Application No. PCT/US2023/027760, filed Jul. 14, 2023, which claims the benefit of and priority to U.S. Provisional Patent Application No. 63/389,155, filed Jul. 14, 2022, the contents of which are incorporated herein by reference.

### BACKGROUND

The ubiquitin proteasome system can be manipulated with different small molecules to trigger targeted degradation of specific proteins of interest. Promoting the targeted degradation of pathogenic proteins using small molecule degraders is emerging as a new modality in the treatment of diseases. One such modality relies on redirecting the activity of E3 ligases such as cereblon (a phenomenon known as E3 reprogramming) using low molecular weight compounds, which have been termed molecular glues to promote the poly-ubiquitination and ultimately proteasomal degradation of new protein substrates involved in the development of diseases. The molecular glues bind to both the E3 ligase and the target protein, thereby mediating an alteration of the ligase surface and enabling an interaction with the target protein.

There exists a need for therapeutics that effectively mediate the degradation of certain proteins for the treatment of diseases, including processes for preparing such therapeutics.

### SUMMARY

Described herein, in part, are compounds contemplated as molecular glues that bind cereblon and mediate the degradation of a protein, and are therefore are useful in the treatment of disorders, such as cancer, and processes for making these compounds. For example, it has been found that compounds of the present disclosure bind to and modulate the surface of cereblon to subsequently mediate the targeted degradation of the protein GSPT1.

In an aspect, disclosed herein is a process for preparing a compound having the structure:

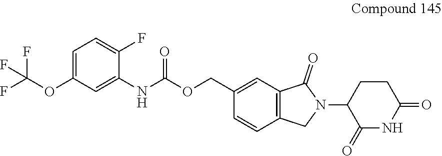 [SMILES: O=C1CCC(N2Cc3ccc(COC(=O)Nc4cc(OC(F)(F)F)ccc4F)cc3C2=O)C(=O)N1]

the process comprising:
   providing Compound 1 having the structure:

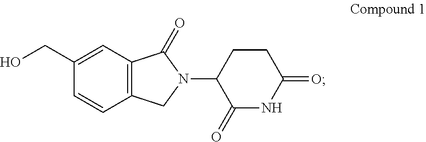 [SMILES: O=C1CCC(N2Cc3ccc(CO)cc3C2=O)C(=O)N1]

providing Compound 1 having the structure:

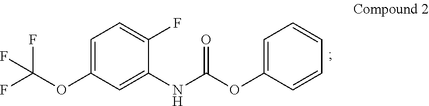 [SMILES: O=C(Nc1cc(OC(F)(F)F)ccc1F)Oc1ccccc1]

and reacting Compound 1 and Compound 2 in the presence of an organic base.

In another aspect, disclosed herein is a crystalline form of a compound having the structure:

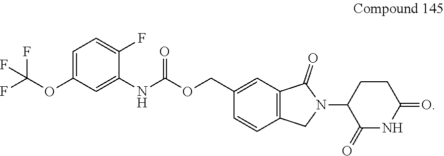 [SMILES: O=C1CCC(N2Cc3ccc(COC(=O)Nc4cc(OC(F)(F)F)ccc4F)cc3C2=O)C(=O)N1]

Also provided herein is a crystalline form of Compound 145 obtainable from the process according to the processes described herein. In an aspect, disclosed herein is a pharmaceutical composition obtainable from a crystalline form of Compound 145, wherein the crystalline form is obtainable from the processes described herein. Further, in an aspect, disclosed herein is a crystalline form of Compound 145, wherein the crystalline form is characterized as having an X-ray powder diffraction pattern as substantially shown in FIG. 1, FIG. 6, or FIG. 7. Also disclosed herein is a crystalline form of Compound 145, wherein the crystalline form is characterized by a powder X-ray diffraction pattern having characteristic peaks in degrees 2θ at 18.8°±0.2°, 22.9°±0.2°, and 23.7°±0.2°, wherein the X-ray diffraction pattern was obtained using Cu Kα radiation.

Also provided herein is a crystalline form of Compound 145, wherein the crystalline form maintains at least about 99 wt % of the compound upon storage under condition of 25° C. at 60% relative humidity for at least 7 days. In an aspect, provided herein is a crystalline form of Compound 145, wherein the crystalline form maintains at least about 99 wt % of the compound upon storage under condition of 40° C. at 75% relative humidity for at least 7 days.

In another aspect, disclosed herein is a pharmaceutical composition comprising the crystalline form described herein (e.g., Form I, Form II or Form III). Further, provided herein is a crystalline form (e.g., Form I, Form II or Form III) obtainable from the processes described herein. For instance, the process comprises crystallizing the crystalline form from ethanol, water, dimethyl sulfoxide, or a combination thereof.

In another aspect, disclosed herein is a pharmaceutical composition obtainable from the crystalline form described herein (e.g., Form I, Form II or Form III), wherein the crystalline form is obtainable from the process described herein.

Methods of treating a cancer in a patient in need thereof, comprising administering to the patient the crystalline form provided herein (e.g., Form I, Form II or Form III), or the pharmaceutical composition provided herein, are also provided.

### BRIEF DESCRIPTION OF THE DRAWINGS

FIG. 1 depicts an exemplary XRPD pattern of compound 145 crystalline Form I.

FIG. 2 depicts an exemplary DSC curve of compound 145 crystalline Form I.

FIG. 3 depicts an overlay of XRPD pattern of crystalline Form I at: day 1 in 40° C./75% RH, day 7 in 40° C./75% RH, day 1 in 25° C./60% RH, and day 7 in 25° C./60% RH.

FIG. 4 depicts an HPLC of crystalline Form I at: day 0, day 1 in 40° C./75% RH, day 7 in 40° C./75% RH, day 1 in 25° C./60% RH, and day 7 in 25° C./60% RH.

FIG. 5 depicts an overlay of XRPD pattern of remained solid in a media (aqueous solution) with about 10 mg of crystalline Form I in 2 mL of media after stirring at 37° C., after 1 hour, 4 hours, and 24 hours.

FIG. 6 depicts an exemplary XRPD pattern of compound 145 crystalline Form II.

FIG. 7 depicts an exemplary XRPD pattern of compound 145 crystalline Form III.

FIG. 8 depicts an exemplary DSC curve of compound 145 crystalline Form III.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Process

The instant disclosure provides a process for preparing a compound having the structure:

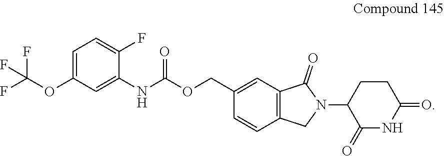 [SMILES: O=C1CCC(N2Cc3ccc(COC(=O)Nc4cc(OC(F)(F)F)ccc4F)cc3C2=O)C(=O)N1]

Specifically, provided herein is a process for preparing a compound having the structure:

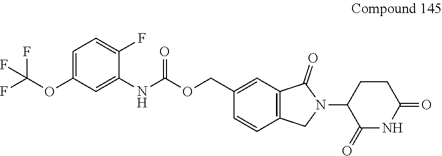 [SMILES: O=C1CCC(N2Cc3ccc(COC(=O)Nc4cc(OC(F)(F)F)ccc4F)cc3C2=O)C(=O)N1]

the process comprising:
   providing Compound 1 having the structure:

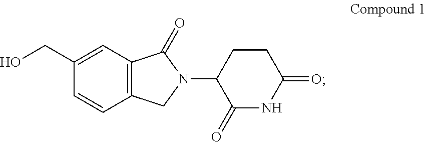 [SMILES: O=C1CCC(N2Cc3ccc(CO)cc3C2=O)C(=O)N1]

providing Compound 1 having the structure:

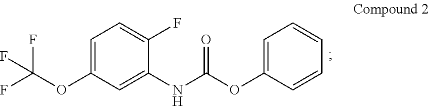 [SMILES: O=C(Nc1cc(OC(F)(F)F)ccc1F)Oc1ccccc1]

and reacting Compound 1 and Compound 2 in the presence of an organic base.

In some embodiments, the organic base is an amine base.

In some embodiments, the amine base is selected from the group consisting of 1,8-diazabicyclo [5.4.0]undec-7-ene (DBU), diisopropylethylamine, and triethylamine.

In some embodiments, the process described herein comprises providing Compound 3 having the structure:

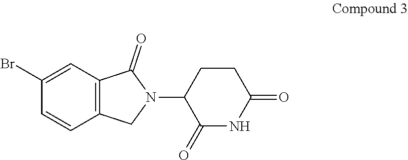 [SMILES: O=C1CCC(N2Cc3ccc(Br)cc3C2=O)C(=O)N1]

and reacting Compound 3 with carbon monoxide in the presence of a catalyst to produce Compound 4 having the structure:

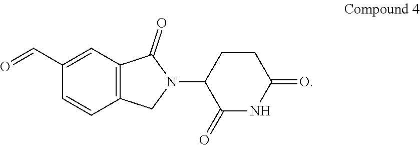 [SMILES: O=Cc1ccc2c(c1)C(=O)N(C1CCC(=O)NC1=O)C2]

In some embodiments, the catalyst is a palladium catalyst.

In some embodiments, the process comprises reacting Compound 3 and carbon monoxide in the presence of a base.

In some embodiments, the process comprises reacting Compound 4 with a borane reactant to produce Compound 1.

In some embodiments, the process comprises providing Compound 5 having the structure:

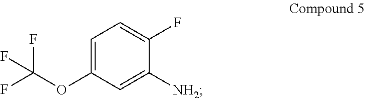 [SMILES: Nc1cc(OC(F)(F)F)ccc1F]

providing Compound 6 having the structure:

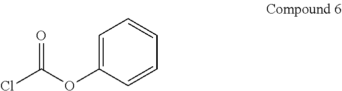

and reacting Compound 5 and Compound 6 in the presence of a base to produce Compound 2.

In some embodiments, the process comprises purifying Compound 145, wherein purifying comprises (i) dissolving Compound 145 in a first solvent to produce a solution and (ii) adding a second solvent to the solution to produce purified Compound 145.

In some embodiments, the process comprises crystallizing the compound (Compound 145) from ethanol, water, dimethyl sulfoxide, or a combination thereof.

The skilled person would understand that Compound 145 has a stereocenter at the carbon atom denoted with “*” as represented by:

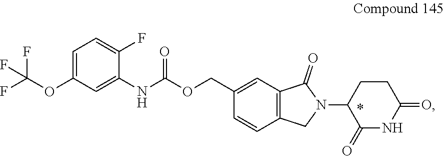 [SMILES: O=C1CCC(N2Cc3ccc(COC(=O)Nc4cc(OC(F)(F)F)ccc4F)cc3C2=O)C(=O)N1]

and wherein “*” denotes a carbon atom that is positioned in either the R or S configuration, as represented by:

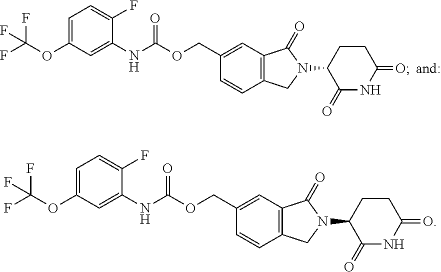

### Crystalline Forms

In certain embodiments, provided herein is a crystalline form of a compound represented by:

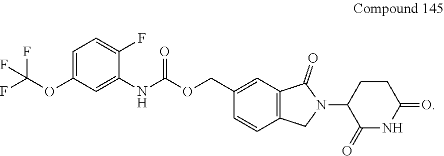 [SMILES: O=C1CCC(N2Cc3ccc(COC(=O)Nc4cc(OC(F)(F)F)ccc4F)cc3C2=O)C(=O)N1]

Also provided herein, in certain embodiments, is a crystalline form obtainable from a process according to any of the processes described herein.

### Form I

In certain embodiments, a crystalline form described herein is an anhydrous crystalline form, Form I. Form I is an anhydrate with high crystallinity. Form I is nonhydroscopic. Form I has good physical and chemical stability when placed in 25° C. 60% RH and 40° C. 75% RH for 7 days. The solubility of Form I in water was less than 0.001 mg/mL

In certain embodiments, the crystalline Form I is obtainable by the processes described herein. For instance, the crystalline Form I is obtainable by the process comprising crystallizing the compound from ethanol, water, dimethyl sulfoxide, or a combination thereof.

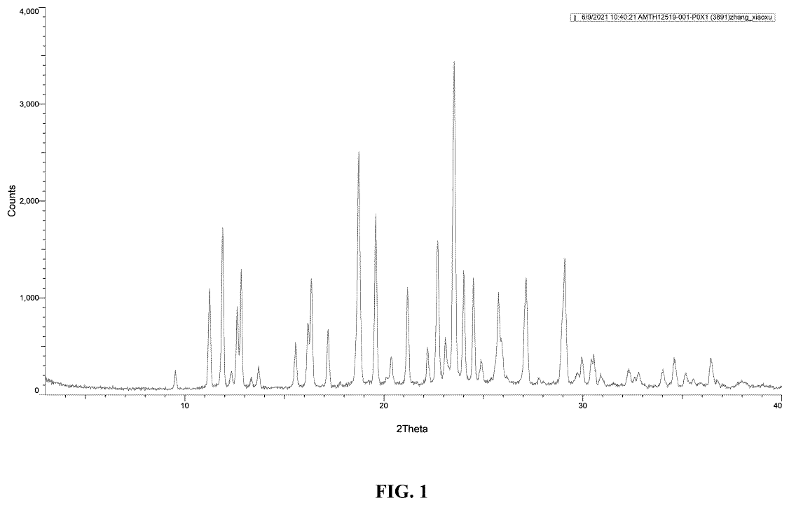

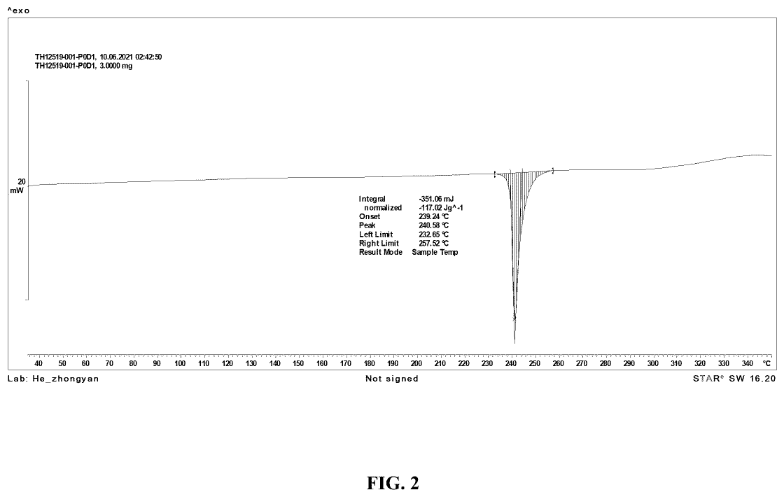

In certain embodiments, the crystalline form is nonhydroscopic. In certain embodiments, the crystalline form is an anhydrous crystalline form, Form I, wherein the crystalline form is characterized by a powder X-ray diffraction pattern having characteristic peaks in degrees 2θ at 11.4°±0.2°, 18.8°±0.2°, 19.7°±0.2°, 22.9°±0.2°, and 23.7°±0.2°, wherein the X-ray diffraction pattern was obtained using Cu Kα radiation. In certain embodiments, the crystalline form is an anhydrous crystalline form, Form I, wherein the crystalline form is characterized by a powder X-ray diffraction pattern having characteristic peaks in degrees 2θ at 11.4°±0.2°, 12.0°±0.2°, 12.9°±0.2°, 16.4°±0.2°, 18.8°±0.2°, 19.7°±0.2°, 22.9°±0.2°, 23.7°±0.2°, 25.9°±0.2°, and 27.3°±0.2.° In various embodiments, the X-ray diffraction pattern was obtained using Cu Kα radiation. In certain embodiments, the crystalline form is an anhydrous crystalline form, Form I, having an X-ray powder diffraction pattern substantially shown in FIG. 1. In certain embodiments, the crystalline form is an anhydrous crystalline form, Form I, having Tₘ substantially shown in FIG. 2. In certain embodiments, the crystalline form is an anhydrous crystalline form, Form I, having Tₘ of about 240° C. by DSC analysis.

In certain embodiments, the crystalline Form I has a solubility less than 0.001 mg/mL at about pH 6 to 7 in an aqueous solution. In some embodiments, the pH is about 6.35, 6.61, or 6.41. In certain embodiments, the crystalline Form I maintains at least about 98 wt % of the compound upon storage under condition of 25° C. at 60% relative humidity for at least 7 days. In certain embodiments, the crystalline Form I maintains at least about 99 wt % of the compound upon storage under condition of 25° C. at 60% relative humidity for at least 7 days. In certain embodiments, the crystalline Form I maintains at least about 98 wt % of the compound upon storage under condition of 40° C. at 75% relative humidity for at least 7 days. In certain embodiments, the crystalline Form I maintains at least about 99 wt % of the compound upon storage under condition of 40° C. at 75% relative humidity for at least 7 days.

In certain embodiments, the crystalline Form I has the compound by about 90% weight or more in crystalline form based on the total weight of the compound present in the crystalline form. In certain embodiments, the crystalline Form I has the compound by about 95% weight or more in crystalline form based on the total weight of the compound present in the crystalline form. In certain embodiments, the crystalline Form I has the compound by about 99% weight or more in crystalline form based on the total weight of the compound present in the crystalline form.

In certain embodiments, provided herein is a stable crystalline form of a compound having the structure:

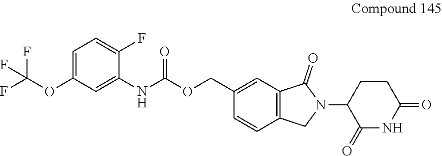 [SMILES: O=C1CCC(N2Cc3ccc(COC(=O)Nc4cc(OC(F)(F)F)ccc4F)cc3C2=O)C(=O)N1]

wherein the stable crystalline form maintains at least about 99 wt % of the compound upon storage under condition of 25° C. at 60% relative humidity for at least 7 days.

In certain embodiments, provided herein is a stable crystalline form of a compound having the structure:

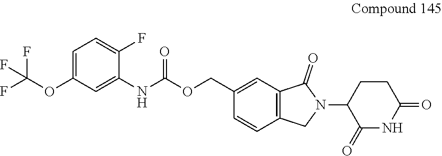 [SMILES: O=C1CCC(N2Cc3ccc(COC(=O)Nc4cc(OC(F)(F)F)ccc4F)cc3C2=O)C(=O)N1]

wherein the stable crystalline form maintains at least about 99 wt % of the compound upon storage under condition of 40° C. at 75% relative humidity for at least 7 days.

### Form II

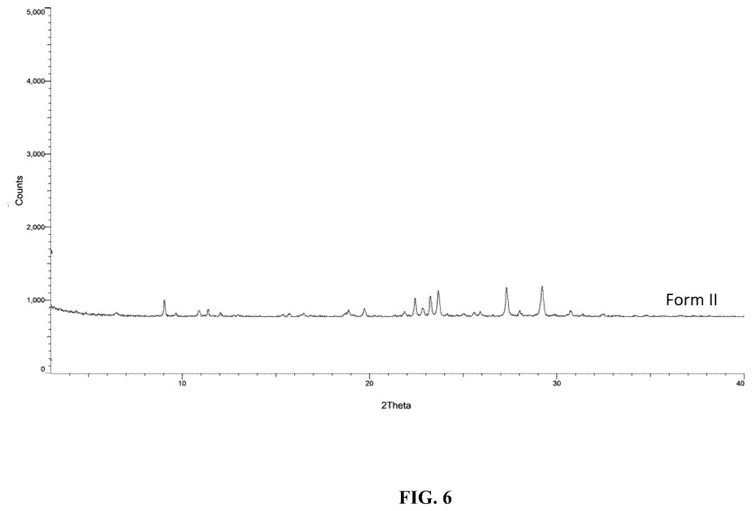

In certain embodiments, the crystalline form is an anhydrous crystalline form, Form II, having an X-ray powder diffraction pattern substantially shown in FIG. 6.

### Form III

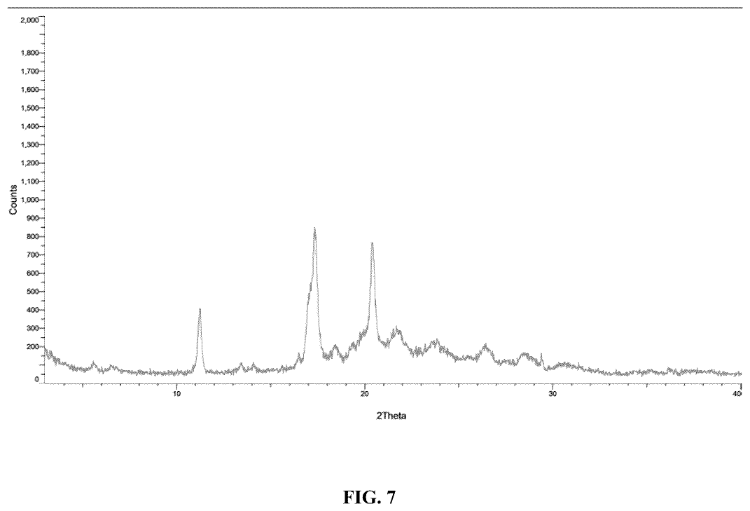

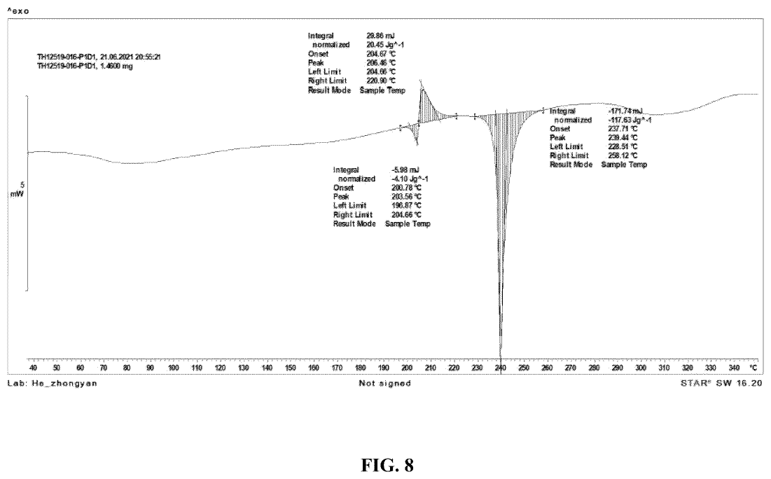

In certain embodiments, the crystalline form is an anhydrous crystalline form, Form III, having an X-ray powder diffraction pattern substantially shown in FIG. 7. In certain embodiments, the crystalline form is an anhydrous crystalline form, Form I, having Tₘ substantially shown in FIG. 8. In certain embodiments, the crystalline form is an anhydrous crystalline form, Form III, having Tₘ of about 203° C. by DSC analysis.

### Pharmaceutical Compositions

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Compositions for oral administration can take the form of bulk liquid solutions or suspensions, or bulk powders. In some embodiments, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component with the remainder being various vehicles or excipients and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component with the remainder being the injectable excipient and the like.

Transdermal compositions are typically formulated as a topical ointment or cream containing the active ingredient(s). When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope of the disclosure provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17^{th} edition, 1985, Mack Publishing Company, Easton, Pennsylvania, which is incorporated herein by reference.

Also provided herein, in certain embodiments, is a pharmaceutical composition comprising a crystalline form described herein and a pharmaceutically acceptable excipient. In certain embodiments, described herein is a pharmaceutical composition obtainable from a crystalline form described herein obtainable from a process according to any the processes described herein.

In certain embodiments, the crystalline form obtainable from a process according to any the processes described herein is an anhydrous crystalline form, Form I. In certain embodiments, the crystalline form is an anhydrous crystalline form, Form I, wherein the crystalline form is characterized by a powder X-ray diffraction pattern having characteristic peaks in degrees 2θ at 18.8°±0.2°, 22.9°±0.2°, and 23.7°±0.2°. In certain embodiments, the crystalline form obtainable from a process according to any the processes described herein is an anhydrous crystalline form, Form I. In certain embodiments, the crystalline form is an anhydrous crystalline form, Form I, wherein the crystalline form is characterized by a powder X-ray diffraction pattern having characteristic peaks in degrees 2θ at 11.4°±0.2°, 18.8°±0.2°, 19.7°±0.2°, 22.9°±0.2°, and 23.7°±0.2°. In certain embodiments, the crystalline form is an anhydrous crystalline form, Form I, wherein the crystalline form is characterized by a powder X-ray diffraction pattern having characteristic peaks in degrees 2θ at 11.4°±0.2°, 12.0°±0.2°, 12.9°±0.2°, 16.4°±0.2°, 18.8°±0.2°, 19.7°±0.2°, 22.9°±0.2°, 23.7°±0.2°, 25.9°±0.2°, and 27.3°±0.2.° In certain embodiments, the crystalline form is an anhydrous crystalline form, Form I, having an X-ray powder diffraction pattern substantially shown in FIG. 1.

Some embodiments described herein relates to a pharmaceutical composition, that can comprise a compound 145 crystalline form, described herein (e.g., crystalline form I, form II, and form III), and a pharmaceutically acceptable carrier, diluent, excipient or combination thereof. In some embodiments, the pharmaceutical composition comprising a crystalline form of any the processes described herein.

In some embodiments, the compound (compound 145) is about 90% to about 95% by weight or more in crystalline Form I based on the total weight of the compound present in the composition. In some embodiments, the compound (compound 145) is about 95% by weight or more in crystalline Form I based on the total weight of the compound present in the composition. In some embodiments, the compound (compound 145) is about 96% by weight or more in crystalline Form I based on the total weight of the compound present in the composition. In some embodiments, the compound (compound 145) is about 97% by weight or more in crystalline Form I based on the total weight of the compound present in the composition. In some embodiments, the compound (compound 145) is about 98% by weight or more in crystalline Form I based on the total weight of the compound present in the composition. In some embodiments, the compound (compound 145) is about 99% by weight or more in crystalline Form I based on the total weight of the compound present in the composition.

In some embodiments, the compound (compound 145) is about 95% by weight or more in crystalline Form II based on the total weight of the compound present in the composition. In some embodiments, the compound (compound 145) is about 96% by weight or more in crystalline Form II based on the total weight of the compound present in the composition. In some embodiments, the compound (compound 145) is about 97% by weight or more in crystalline Form II based on the total weight of the compound present in the composition. In some embodiments, the compound (compound 145) is about 98% by weight or more in crystalline Form II based on the total weight of the compound present in the composition. In some embodiments, the compound (compound 145) is about 99% by weight or more in crystalline Form II based on the total weight of the compound present in the composition.

In some embodiments, the compound (compound 145) is about 95% by weight or more in crystalline Form III based on the total weight of the compound present in the composition. In some embodiments, the compound (compound 145) is about 96% by weight or more in crystalline Form III based on the total weight of the compound present in the composition. In some embodiments, the compound (compound 145) is about 97% by weight or more in crystalline Form III based on the total weight of the compound present in the composition. In some embodiments, the compound (compound 145) is about 98% by weight or more in crystalline Form III based on the total weight of the compound present in the composition. In some embodiments, the compound (compound 145) is about 99% by weight or more in crystalline Form III based on the total weight of the compound present in the composition.

### Methods of Treatment

The compounds of the disclosure, e.g., a crystalline form described herein, modulate the activity of cereblon. Thus, the compounds and compositions of the disclosure can be useful as a medicament, i.e. as a medicament in therapy, more specifically for the treatment of cancer, as detailed below. Therefore, in a further aspect, the present disclosure provides a method of treatment of a mammal, for example, a human, suffering from cancer, as detailed below. The term “treatment” is intended to encompass prophylaxis, therapy and cure. Such treatment comprises the step of administering a therapeutically effective amount of a compound of Formula I or salt thereof (or of a pharmaceutical composition containing a compound of Formula I or salt thereof) to said mammal, for example, a human.

Thus, the disclosure is directed towards the use of the compounds, including crystalline forms, of the disclosure or pharmaceutically acceptable salts or stereoisomers thereof or a pharmaceutical composition thereof for the treatment of a disease associated or caused with GSPT1, in particular the treatment of cancer, as detailed below, in a mammal, for example a human.

### Myc-driven Cancers

Described herein, in some embodiments, are cancers exhibiting increased expression of one or more of c-Myc, L-Myc, N-Myc, EIF4EBP1, and EIF4EBP2 as well as ones with increase phosphorylation of one or both of EIF4EBP1 and EIF4EBP2.

Myc-driven cancers refer to cancers where there is abnormal activation of Myc oncogene, either due to transcriptional overexpression (e.g., caused by gene amplification, translocation, alterations in upstream signaling pathways) and/or protein stabilization. A myc-driven cancer cell includes a cancer cell that has an increased expression or overexpression (and/or increased activity) of at least one myc transcription factor such as N-myc and/or L-myc and/or C-myc, or a surrogate marker thereof, relative to a control cell such as a normal (e.g., non-cancerous) cell of the same or corresponding cell type. The term “cancerous” when referring to a sample such as a cell or tissue, generally refers to any sample, such as cells or tissues that exhibit, or are predisposed to exhibiting, unregulated growth, including, for example, a neoplastic cell/tissue such as a premalignant cell/tissue or a cancer cell (e.g., carcinoma cell or sarcoma cell).

In some embodiments the Myc-driven cancer or tumor as defined herein refers to a blood borne tumor cancer, such as a hematological cancer, preferably a cancer of hematopoietic and lymphoid tissues and lymphatic system, such as blood cancer, bone marrow cancer, lymph node cancer, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, non-Hodgkin's lymphomas and multiple myeloma (MM).

In some embodiments, the Myc-driven cancer or tumour is a solid tumor cancer, such as breast cancer, colorectal cancer, lung cancer, e.g. SCLC, NSCLC, neuroendocrine cancer, e.g., neuroendocrine prostate cancer (for example, NEPC (castration-resistant neuroendocrine prostate cancer)) and lung neuroendocrine tumors (Lu-NETs), liver cancer, stomach cancer, pancreatic cancer, gastric cancer, esophageal cancer, bladder cancer, skin cancer, brain cancer, cervical cancer, ovarian cancer, melanoma and head and neck cancer.

In some embodiments the Myc-driven cancer as used herein refers in particular to breast cancer and SCLC. In some embodiments the myc-driven cancer as used herein refers in particular to NSCLC. In some embodiments, the cancer is solid tumor cancer exhibiting amplification of the N-Myc gene and/or the L-Myc gene. In some embodiments the Myc-driven cancer as used herein refers to neuroendocrine cancer, for example, neuroendocrine prostate cancer (for example, NEPC (castration-resistant neuroendocrine prostate cancer)) and lung neuroendocrine tumors (Lu-NETs), acute myelogenous leukemia (AML), lymphoma, and multiple myeloma (MM).

### Solid and Liquid Cancers

The term “solid cancer” or “solid tumor” refers to disease of tissues or organs, such as to malignant, neoplastic, or cancerous solid tumors, i.e. sarcomas, carcinomas. The tissue structure of solid tumors includes interdependent tissue compartments and usually does not contain cysts or fluid areas. A solid cancer or solid tumor includes cancers of the bladder, bone, brain, breast, cervix, chest, colon, endrometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer (for example, NEPC (castration-resistant neuroendocrine prostate cancer)) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, cervical cancer, ovarian cancer, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In some embodiments, a solid cancer or solid tumor is a cancer of the breast, lung, stomach, colon, bladder, brain, pancreas, liver, head and neck, prostate, ovaries, upper aerodigestive tract and the like.

The term “blood borne cancer” or “blood borne tumor” (also typically referred to as “hematological cancer”) refers to cancer of the body's blood-forming and immune system—the bone marrow and lymphatic tissue. The tissue structure of blood-borne cancers or tumors includes an abnormal mass of cells that is fluid in nature. Such cancers include leukemias (malignant neoplasms of the blood-forming tissues), lymphomas (Non-Hodgkin's Lymphoma), Hodgkin's disease (Hodgkin's Lymphoma) and myeloma. In one embodiment, the myeloma is multiple myeloma (MM). In some embodiments, the leukemia is, for example, acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, or B-cell acute lymphoblastic leukemia. The leukemia can be relapsed, refractory or resistant to conventional therapy. In some embodiments, the lymphoma is, for example, diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma. In one embodiment, the hematological cancer is indolent lymphoma including, for example, DLBCL, follicular lymphoma, or marginal zone lymphoma. In some embodiments blood-borne cancers or hematological cancers include acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, non-Hodgkin's lymphomas and multiple myeloma (MM).

In particular embodiments, the compounds of the disclosure, e.g., a crystalline form of a compound described herein, or pharmaceutically acceptable salts or stereoisomers thereof or a pharmaceutical composition thereof are used for the treatment of cancer associated with GSPT1, such as solid cancers including but not limited to cancers of the bladder, bone, brain, breast, cervix, chest, colon, endrometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, uterus, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMOL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

Such a use (or method of treatment) of a subject comprises administering to a subject in need of such treatment a therapeutically effective amount of a compound of the disclosure or pharmaceutically acceptable salts thereof or a pharmaceutical composition thereof by targeting cereblon.

Disclosed herein, in part, is a method of treating a Myc-driven cancer in a subject in need thereof, comprising administering the subject a therapeutically effective amount of a compound described herein or a composition as described herein.

In some embodiments, the Myc-driven cancer is a Myc-driven lung cancer.

In some embodiments, the Myc-driven cancer is characterized by high driven Myc tumor.

In some embodiments, the Myc-driven cancer is a Myc-driven small cell lung cancer.

In some embodiments, the Myc-driven small cell lung cancer is a high L-Myc small cell lung cancer.

In some embodiments, the Myc-driven cancer is a Myc-driven non-small cell lung cancer.

In some embodiments, the Myc-driven non-small cell lung cancer is a high N-Myc non-small cell lung cancer.

In some embodiments, the compound or the composition is administered to the subject via oral administration.

In another aspect, provided herein is a method of degrading GSPT1 in a subject suffering from cancer, comprising administering to the subject a therapeutically effective amount of a compound described herein or a composition described herein.

In some embodiments, the cancer is a Myc-driven cancer.

In some embodiments, the Myc-driven cancer is a Myc-driven lung cancer.

In some embodiments, the Myc-driven cancer is a Myc-driven small cell lung cancer.

In some embodiments, the Myc-driven small cell lung cancer is a high L-Myc small cell lung cancer.

In some embodiments, the Myc-driven cancer is a Myc-driven non-small cell lung cancer.

In some embodiments, the Myc-driven non-small cell lung cancer is a high N-Myc non-small cell lung cancer.

In some embodiments, the compound or the composition is administered to the subject via oral administration.

In another aspect, the disclosure is directed to a method of reducing the level of GSPT1 in a subject suffering from cancer, comprising administering the subject a therapeutically effective amount of a compound or a composition as described herein.

In some embodiments, the cancer is a Myc-driven cancer.

In some embodiments, the Myc-driven cancer is a Myc-driven lung cancer.

In some embodiments, the Myc-driven cancer is a Myc-driven small cell lung cancer.

In some embodiments, the Myc-driven small cell lung cancer is a high L-Myc small cell lung cancer.

In some embodiments, the Myc-driven cancer is a Myc-driven non-small cell lung cancer.

In some embodiments, the Myc-driven non-small cell lung cancer is a high N-Myc non-small cell lung cancer.

In some embodiments, the compound or the composition is administered to the subject via oral administration.

### Definitions

Definitions of specific functional groups and chemical terms are described in more detail below.

Unless defined otherwise, all technical and scientific terms used herein have the same meaning as commonly understood by one of ordinary skill in the art to which this invention belongs. Unless defined otherwise, all abbreviations used herein have their conventional meaning within the chemical and biological arts. The chemical structures and formulae set forth herein are constructed according to the standard rules of chemical valency known in the chemical arts.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from the group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The articles “a” and “an” are used in this disclosure to refer to one or more than one (i.e., at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “comprise,” “comprises,” “comprising,” “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

The term “pharmaceutical composition” refers to a mixture of one or both compounds disclosed herein with other chemical components, such as diluents or carriers. The pharmaceutical composition facilitates administration of the compound to an organism. Pharmaceutical compositions will generally be tailored to the specific intended route of administration.

The term “pharmaceutically acceptable” defines a carrier, diluent, excipient, salt or composition that is safe and effective for its intended use and possesses the desired biological and pharmacological activity.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability, retarded dissolution etc., to the composition. A “diluent” is a type of excipient.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The terms “stable” and “stability” herein means that a pharmaceutical composition is stable, for example, with respect to heat, light, temperature, and/or humidity.

As used herein, an “assay” refers to a specific, stability-indicating procedure that determines the content of the drug substance. For example, an assay can be a chromagraphic method (e.g., HPLC) involving use of a reference standard.

As used herein, “crystalline” refers to a solid having a highly regular chemical structure, i.e., having long range structural order in the crystal lattice. The molecules are arranged in a regular, periodic manner in the 3-dimensional space of the lattice. In particular, a crystalline form may be produced as one or more single crystalline forms.

### EXAMPLES

Procedures for making compounds described herein are provided below. Starting materials used in the following schemes can be purchased or prepared by methods described in the chemical literature, or by adaptations thereof, using methods known by those skilled in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated.

Abbreviations: BH3-THF=borane-tetrahydrofuran complex; CO=carbon monoxide; DIPEA=N,N-Diisopropylethylamine; DMAc=dimethylacetamide; DMF=dimethylformamide; DMSO=dimethyl sulfoxide; EtOH=ethanol; K2CO3=potassium carbonate; MeTHF=2-methyltetrahydrofuran; Pd(dppf)Cl₂=[1,1′-bis(diphenylphosphino) ferrocene]-dichloro-palladium(II); THF=tetrahydrofuran.

### Example 1. Synthesis of Compound 145

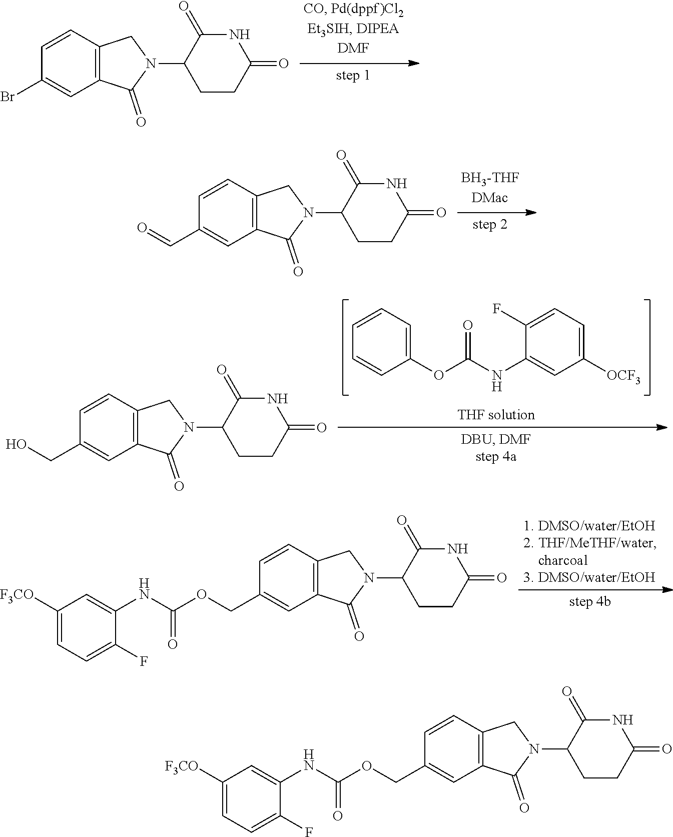

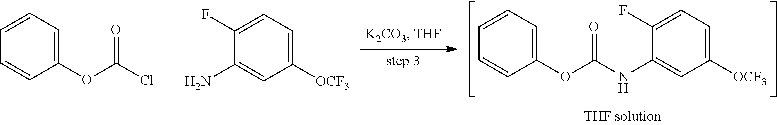

Step 1. To a solution of dimethyl formamide (DMF), diisopropylethyl amine is added palladium catalyst, Pd(dppf)Cl2 and the substrate 3-(6-bromo-1-oxoisoindolin-2-yl) piperidine-2,6-dione. After stirring at room temperature (r.t.), triethylsilane (Et3SiH) is added and a pressure of carbon monoxide is applied at elevated temperatures until conversion is complete by in process control (IPC)-1.1. The reaction mixture is cooled down to r.t., water and seed crystals are added, and the mixture is left stirring at r.t. for few hours. Crystallization is completed by addition of ethyl acetate (EtOAc), cooling to 2 to 8° C., and stirring for few hours. Product is isolated by filtration washing with ethanol (EtOH) as crude and dried under vacuum. The product is purified by suspending in ethanol (EtOH) at r.t., and then collecting the solids by filtration. Drying is performed under vacuum until ethanol (EtOH) level is reached by IPC-1.2.

Step 2. Borane-tetrahydrofuran complex (BH3-THF) is added to a mixture of 2-(2,6-dioxopiperidin-3-yl)-3-oxoisoindoline-5-carbaldehyde in dimethylacetamide at r.t. The resulting mixture is stirred until complete conversion by IPC-2.1. The reaction mixture is quenched by methanol (MeOH) addition, concentrated under vacuum, and diluted with ethanol (EtOH) to complete crystallization. Crude product is isolated by filtration, and it is further slurried in ethyl acetate (EtOAc). After filtration, purified product is obtained and dried under reduced pressure until residual ethanol (EtOH) limit was reached by IPC-2.2.

Step 3. To a suspension of 2-fluoro-5-(trifluoromethoxy) aniline and potassium carbonate (K2CO3) in tetrahydrofuran (THF), phenyl carbonochloridate is slowly added at r.t. After conversion limit is achieved by IPC-3.1, the mixture is filtered, and the filtrate is concentrated under reduced pressure to yield phenyl (2-fluoro-5-(trifluoromethoxy)phenyl)carbamateas concentrated solution in THF, which is stored briefly and used as such.

Step 4a. The prepared THF solution of phenyl (2-fluoro-5-(trifluoromethoxy)phenyl)carbamate from step 3 is slowly added to a solution of 3-(6-(hydroxymethyl)-1-oxoisoindolin-2-yl) piperidine-2,6-dione and diazabicycloundec-7-ene (DBU) in DMF at r.t. Stirring was continued until IPC-4.1 showed consumption of 3-(6-(hydroxymethyl)-1-oxoisoindolin-2-yl) piperidine-2,6-dione. The resulting mixture is quenched into a diluted aqueous solution of HCl. After leaving at r.t., the crude product (2-(2,6-dioxopiperidin-3-yl)-3-oxoisoindolin-5-yl) methyl (2-fluoro-5-(trifluoromethoxy)phenyl)carbamate is collected by filtration. The wet filter cake is dissolved in THF, and the organic layer is washed with aqueous 10% N-acetyl cysteine/sodium carbonate (Na2CO3) solution. After back-extraction of the aqueous phase with THF, the combined organic phases are washed with brine. The resulting organic layer is filtered and concentrated under vacuum. 2-Methyl tetrahydrofuran (MeTHF) and water are added, and the suspension stirred at r.t. After filtration and drying, (2-(2,6-dioxopiperidin-3-yl)-3-oxoisoindolin-5-yl) methyl (2-fluoro-5-(trifluoromethoxy)phenyl)carbamate is obtained as crude product, IPC-4.2.

Step 4b. To a solution of crude (2-(2,6-dioxopiperidin-3-yl)-3-oxoisoindolin-5-yl) methyl (2-fluoro-5-(trifluoromethoxy)phenyl)carbamate in dimethyl sulfoxide (DMSO), a mixture of ethanol/water is added at r.t. followed by seeding. After stirring at r.t., additional ethanol/water is charged. The suspension is filtered, and the wet cake is slurried with ethanol (EtOH). Filtration and drying provided (2-(2,6-dioxopiperidin-3-yl)-3-oxoisoindolin-5-yl)methyl (2-fluoro-5-(trifluoromethoxy)phenyl)carbamate, IPC-4.3.

### Example 2. Manufacturing Procedure of Compound 145

### Overall Scheme

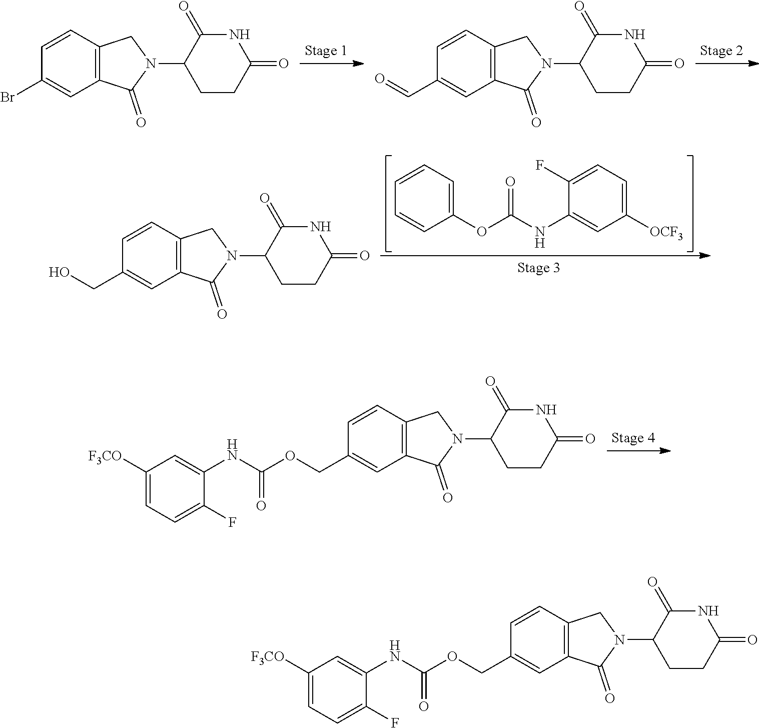

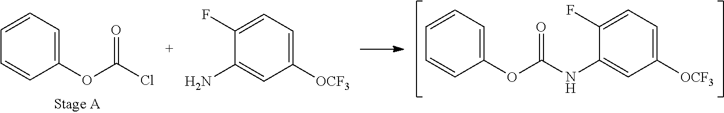

### Stage 1.

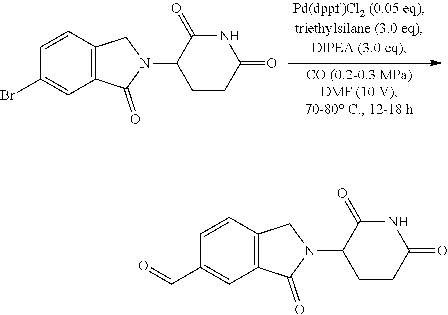 [SMILES: O=C1CCC(N2Cc3ccc(Br)cc3C2=O)C(=O)N1 ; O=Cc1ccc2c(c1)C(=O)N(C1CCC(=O)NC1=O)C2]

Preparation: 0.19 kg of water was charged into 534 kg DMF to give wet DMF (356 ppm water). DIPEA (35 kg, 1.2×, 3.0 eq), Pd(dppf)Ch (3.28 kg, 0.113×, 0.0 Seq) and 3-(6-bromo-1-oxoisoindolin-2-yl) piperidine-2,6-dione (28.9 kg, 1.0×, 1.0 eq) were charged into wet DMF (151 kg, 5.5 vol), and then rinsed with wet DM F (182 kg, 6.7 vol). The temperature was adjusted to 15-25° C. and stirred at 15-25° C. for 0.5 h. Et₃SiH (31 kg, 1.1×, 3.0 eq) was charged and rinsed with wet DMF (8 kg, 0.3 vol), set to vacuum mode to ≤−0.08 MPa then refill with CO to 0.3-1.1 MPa. The temperature was adjusted to 85-91° C. and stirred at 85-91° C. for 18-30 h until analysis shows the reaction is complete. The temperature was adjusted to 10-20° C., the resulting mixture was filtered, and the wet cake was washed with wet DMF twice (42 kg+38 kg, 1.5+1.3 vol). The filtrate was concentrated to 2-4 vol at 50-60° C. under vacuum for 46 h (vacuum degree was not recorded, solid precipitation over concentration), adjusted to 10-20° C. and charged with EtOAc (286 kg, 11 vol) at 10-20° C. and stirred at 10-20° C. for 4 h. The resulting mixture was filtered, and the wet cake was washed with EtOAc (23 kg, 0.9 vol). The resulting residue was dried at 45-55° C. under vacuum for 36 h to give 2-(2,6-dioxopiperidin-3-yl)-3-oxoisoindoline-5-carbaldehyde (19 kg, assay: 15.7%, yield: 12.3%).

Purification: 2-(2,6-Dioxopiperidin-3-yl)-3-oxoisoindoline-5-carbaldehyde (1.0×, 1.0 eq) was charged into EtOH (10-11 vol), and then stirred at 20-30° C. for 2 h. The resulting residue was filtered and washed with EtOH (0.6-0.8 vol). The wet cake in EtOAc (5 vol) was slurried at 20-30° C. for 2 h, filtered and washed with EtOAc (0.3-0.8 vol). The residue was dried at 50-60° C. under vacuum for 20 h to give purified 2-(2,6-dioxopiperidin-3-yl)-3-oxoisoindoline-5-carbaldehyde (95% yield).

### Stage 2.

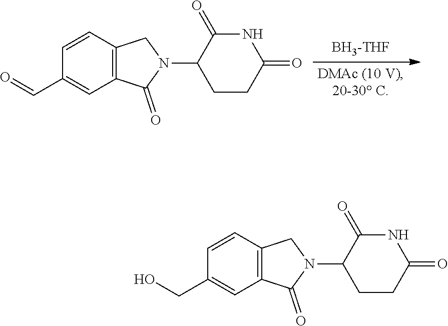 [SMILES: O=Cc1ccc2c(c1)C(=O)N(C1CCC(=O)NC1=O)C2 ; O=C1CCC(N2Cc3ccc(CO)cc3C2=O)C(=O)N1 ; "BH3-THF"]

2-(2,6-Dioxopiperidin-3-yl)-3-oxoisoindoline-5-carbaldehyde (1.0×, 1.0 eq) was dissolved in DMAc (9-10 vol) and adjusted to 20-30° C. BH₃·THF (1M in THF, 1.0-1.2 eq) was charged at 20-30° C. within 1.5 h (mild exthermic), and the resulting mixture was stirred at 20-30° C. for 1-2 h until analysis showed the reaction is complete. The reaction mixture was quenched with MeOH (2 vol, 13 eq) at 20-30° C. within 2 h (strong exthermic and gas evolution) and stirred at 20-30° C. for 13-16 h. The resluting solution was concentrated to 3-4 vol at 55-65° C. under vacuum at ≤−0.1 MPa (solid precipitate during concentration), and then adjusted to 20-30° C. EtOH (10 vol) was charged at 20-30° C. and stirred at 20-30° C. for 3-4 h. The resulting mixture was filtered and the wet cake was washed with EtOH (1 vol), and the wet cake was dried at 50-60° C. under vacuum for 16 h to give crude 3-(6-(hydroxymethyl)-1-oxoisoindolin-2-yl) piperidine-2,6-dione. Slurry the crude product in EtOAc (4-5 vol) at 20-30° C. for 14 h, the resulting mixture was filtered and the wet cake was washed with EtOAc (0.5 vol) (Slurry to reduce Pd content, the color of solid turned from black to brown after slurry and the mother liquid is black, Pd content data were not collected.). The wet cake was dried at 55-65° C. under vacuum for 17 h to give 3-(6-(hydroxymethyl)-1-oxoisoindolin-2-yl) piperidine-2,6-dione (94% yield).

### Stage 3

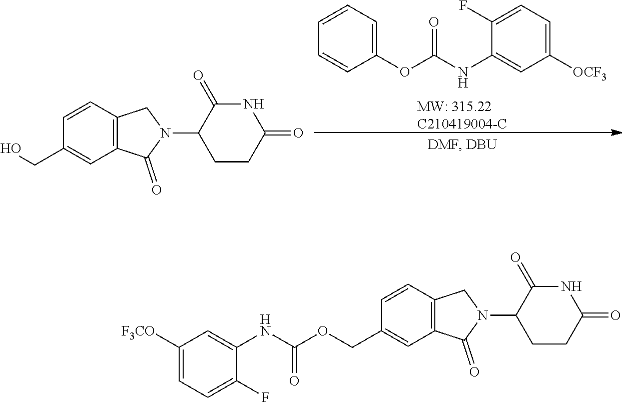

DMF (8 vol), 3-(6-(hydroxymethyl)-1-oxoisoindolin-2-yl) piperidine-2,6-dione (1×) and DBU (1.7×, 3.0 eq) were charged into R1. R1 was adjusted to 10-20° C. and phenyl (2-fluoro-5-(trifluoromethoxy)phenyl)carbamate in THF (1.5 eq theory amount, refer to Stage 5. 1.7× net amount, contains 10-20 wt % THF) was charged to R1 within 2 h (mild exthermic). R1 was stirred for 1-3 h at 10-20° C. until HPLC showed the reaction is complete. The content of R1 was charged into aq. HCl (3.3 eq in 20 vol water) at 15-25° C. within 2 h (mild exthermic). The resulting mixture was stirred for 6-12 hr at 15-25° C. The cake was filtered and washed with H₂O (2 vol).

Wet (2-(2,6-dioxopiperidin-3-yl)-3-oxoisoindolin-5-yl) methyl (2-fluoro-5-(trifluoromethoxy)phenyl)carbamate was charged into THF (25 vol) and then washed with aq. N-acetylcysteine/Na₂CO₃ (1× N-acetylcysteine and 1× Na₂CO₃ pre-dissolved in 10 vol H₂O) at 20-30° C. The aqueous layer was extracted with THF (8 vol) at 20-30° C. The organic layer was combined and washed with aq. 10% N-acetylcysteine/10% Na₂CO₃ (5 vol). The resulting mixture was then washed with saturated NaCl (5 vol) 2-3 times. SiliaMetS® was charged into the organic layer (a clear solution, ˜32 vol) and stirred at 15-30° C. for 1 or 14 h. The cake was filtered over diatomite (0.5×) and washed with-THF/H₂O (V/V=20:1) (5 vol). The filtrate was concentrated below 40° C. under vacuum to 2-4 vol. 2-MeTHF (8 vol) and H₂O (10 vol) were charged, stirred at 20-30° C. for 0.5 h. The mixture was filtered and the wet cake was washed with 2-MeTHF (1 vol) and dried at 25-55° C. under vacuum to give crude (2-(2,6-dioxopiperidin-3-yl)-3-oxoisoindolin-5-yl) methyl (2-fluoro-5-(trifluoromethoxy)phenyl)carbamate (80.4% yield).

### Stage 4

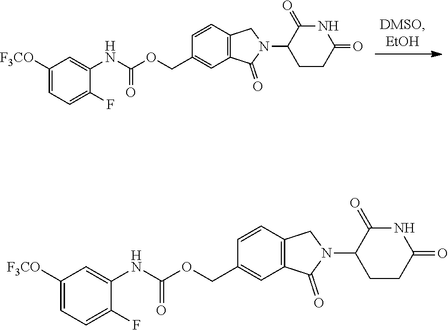 [SMILES: O=C1CCC(N2Cc3ccc(COC(=O)Nc4cc(OC(F)(F)F)ccc4F)cc3C2=O)C(=O)N1 ; O=C1CCC(N2Cc3ccc(COC(=O)Nc4cc(OC(F)(F)F)ccc4F)cc3C2=O)C(=O)N1]

The crude product was dissolved in DMSO (5 vol), filtered over diatomite (0.5×) into the reactor and H₂O (5 vol) was charged within 3 h at 15-25° C. The resulting mixture was stirred at 15-25° C. for 1 h and then filtered. The wet cake was reslurried in DMSO (2 vol) at 45-55° C. for 2 h, adjusted to 15-25° C. slowly and then stirred at 15-25° C. for 10 h. EtOH (15 vol) was charged within 3.5 h (slowly charge to avoid the entrap of DMSO) at 20-30° C. and stirred at 20-30° C. for 3 h. The wet cake was filtered and reslurried in EtOH (10 vol) at 45-55° C. for 8 h, adjusted to 20-30° C. within 4 h and stirred at 20-30° C. for 8 h. The resulting mixture was filtered and the wet cake was reslurried in EtOH (14 vol) at 45-55° C. for 8 h, adjusted to 20-30° C. within 4 h and stirred at 20-30° C. for 9 h. The resulting mixture was filtered and the wet cake was reslurried in DMSO (1.6 vol) at 45-55° C. for 8 h, then adjusted to 20-30° C. slowly. EtOH (11.8 vol) was charged slowly at 20-30° C. and stirred at 20-30° C. for 0.5 h. The resulting mixture was filtered and the wet cake was washed with EtOH (2 vol). The wet cake was reslurried in EtOH (12.5 vol) at 45-55° C. for 8 h, adjusted to 20-30° C. slowly and stirred at 20-30° C. for 1 h. The resluting mixture was filtered, the wet cake was washed with EtOH (2 vol) and dried at 45-55° C. for 25 h under vacuum to give (2-(2,6-dioxopiperidin-3-yl)-3-oxoisoindolin-5-yl) methyl (2-fluoro-5-(trifluoromethoxy)phenyl)carbamate (73.7% yield).

### Stage A

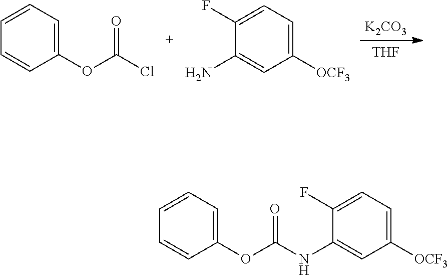

2-Fluoro-5-(trifluoromethoxy) aniline (1.0×, 1 eq), K₂CO₃ (1.3 eq) was charged into THF (10 vol), adjusted to 15-25° and phenyl chloroformate (0.8×, 1.0 eq) was charged at 15-25° within 1 h (mild exthermic). The resulting mixture was stirred at 15-25° C. for 4 h until analysis showed the reaction is complete. The resulting mixture was filtered and the wet cake was washed with THF (2 vol). The filtrate was concentrated to 1-2 vol below 40° C. under vacuum, and the crude phenyl (2-fluoro-5-(trifluoromethoxy)phenyl)carbamate in THF was used directly to the next step.

### Example 2A. Manufacturing Procedure of Compound 145

### Overall Scheme

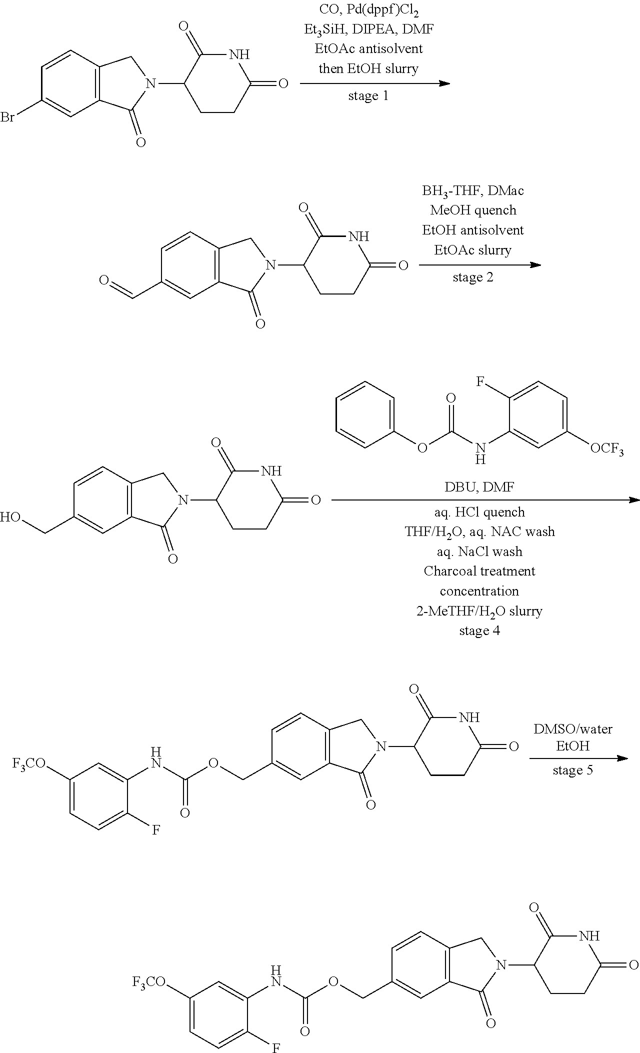 [SMILES: O=C1CCC(N2Cc3ccc(Br)cc3C2=O)C(=O)N1 ; O=Cc1ccc2c(c1)C(=O)N(C1CCC(=O)NC1=O)C2 ; O=C1CCC(N2Cc3ccc(CO)cc3C2=O)C(=O)N1 ; O=C(Nc1cc(OC(F)(F)F)ccc1F)Oc1ccccc1 ; O=C1CCC(N2Cc3ccc(COC(=O)Nc4cc(OC(F)(F)F)ccc4F)cc3C2=O)C(=O)N1 ; O=C1CCC(N2Cc3ccc(COC(=O)Nc4cc(OC(F)(F)F)ccc4F)cc3C2=O)C(=O)N1]

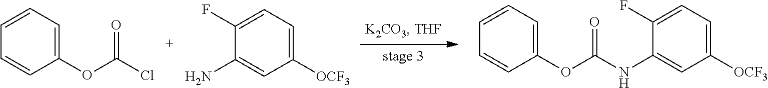 [SMILES: O=C(Cl)Oc1ccccc1 ; Nc1cc(OC(F)(F)F)ccc1F ; O=C(Nc1cc(OC(F)(F)F)ccc1F)Oc1ccccc1]

### Stage 1.

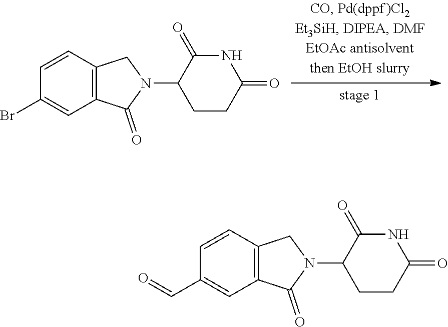 [SMILES: O=C1CCC(N2Cc3ccc(Br)cc3C2=O)C(=O)N1 ; O=Cc1ccc2c(c1)C(=O)N(C1CCC(=O)NC1=O)C2]

### Exemplary Preparation 1 of 2-(2,6-dioxopiperidin-3-yl)-3-oxoisoindoline-5-carbaldehyde

To a solution of DMF (89 kg, 8.1 wt), DIPEA (13.2 kg, 1.2 wt, 3.0 eq) is added Pd(dppf)Cl₂ (1.10 kg, 0.1 wt, 0.44 eq) and the substrate 3-(6-bromo-1-oxoisoindolin-2-yl) piperidine-2,6-dione (11.0 kg, 1.0 wt, 1.0 eq). After stirring at 15-25° C. for 0.5 h, Et₃SiH (11.9 kg, 1.1 wt, 3.0 eq) is added and then pressurized with carbon monoxide (0.1-0.5 MPa, controlled at 0.2-0.3 MPa during production) and heated at 70-80° C. for 19 h (homogeneous). The reaction mixture is cooled down to 20-30° C., process water (0.13 kg, 0.01 wt, 0.2 eq) is charged then DMF (4 kg) is charged to rinse the pipeline (slow down the reaction to better control the end point). Pressurized with carbon monoxide (0.1-0.5 MPa, controlled at 0.2-0.3 MPa during production) and heated at 70-80° C. for until the reaction is complete (solid out). The reaction mixture is cooled down to 20-30° C., process water (0.7 kg, 0.06 wt, 1.1 eq) is charged then DMF (3 kg) is charged to rinse the pipeline. Seed is added (0.56 kg, 0.05 wt) and aging at 20-30° C. for 1 h. After the addition of EtOAc in 1 h (221 kg, 20.1 wt), the reaction mixture is cooled to −5-5° C. within 2 h and stir at −5-5° C. for 10 h. The solid is collected by filtration as crude after washing with EtOH (40 kg, 3.6 wt) and dried under vacuum at 45-55° C. for 24 h. The product was slurried in EtOH (28 kg, 2.5 wt) at 20-30° C. for 5 h. The solid is collected by filtration after washing with EtOH (10 kg, 0.9 wt). Drying is performed under vacuum at 45-55° C. for 24 h to give 2-(2,6-dioxopiperidin-3-yl)-3-oxoisoindoline-5-carbaldehyde as off-white solid (6.14 kg with 86.8% assay in 57% yield from starting material).

### Exemplary Preparation 2 of 2-(2,6-dioxopiperidin-3-yl)-3-oxoisoindoline-5-carbaldehyde

To a solution of DMF (86 kg, 7.9 wt), DIPEA (13.2 kg, 1.2 wt, 3.0 eq) is added Pd(dppf)Cl₂ (1.10 kg, 0.1 wt, 0.44 eq) and the substrate 3-(6-bromo-1-oxoisoindolin-2-yl) piperidine-2,6-dione (10.9 kg, 1.0 wt, 1.0 eq). After stirring at 15-25° C. for 0.5 h, Et₃SiH (11.6 kg, 1.1 wt, 2.9 eq) is added and then pressurized with carbon monoxide (0.1-0.5 MPa, controlled at 0.2-0.3 MPa during production) and heated at 70-80° C. for 14 h (homogeneous). The reaction mixture is cooled down to 20-30° C., process water (0.12 kg, 0.01 wt, 0.2 eq) is charged then DMF (2 kg) is charged to rinse the pipeline (slow down the reaction to better control the end point). Pressurized with carbon monoxide (0.1-0.5 MPa, controlled at 0.2-0.3 MPa during production) and heated at 70-80° C. until the reaction is complete (solid out). The reaction mixture is cooled down to 20-30° C., process water (0.6 kg, 0.06 wt, 0.21.0 eq) is charged then DMF (2 kg) is charged to rinse the pipeline. Seed is added (0.75 kg, 0.07 wt) and aging at 20-30° C. for 1 h. After the addition of EtOAc in 1.5 h (211 kg, 19.4 wt), the reaction mixture is cooled to −5-5° C. within 1.5 h and stir at −5-5° C. for 10 h. The solid is collected by filtration as crude after washing with EtOH (22 kg, 2 wt) and dried under vacuum at 45-55° C. for 24 h. The product was slurried in EtOH (26 kg, 2.4 wt) at 20-30° C. for 6 h. The solid is collected by filtration and after washing with EtOH wash (9 kg, 0.8 wt). Drying is performed under vacuum at 45-55° C. for 20 h to give 2-(2,6-dioxopiperidin-3-yl)-3-oxoisoindoline-5-carbaldehyde as off-white solid (6.58 kg with 88.7% assay in 64% yield from starting material).

### Stage 2.

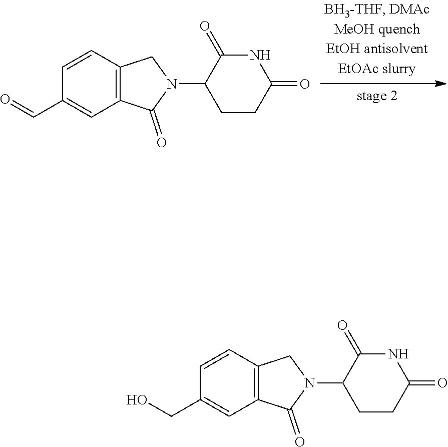 [SMILES: O=Cc1ccc2c(c1)C(=O)N(C1CCC(=O)NC1=O)C2 ; O=C1CCC(N2Cc3ccc(CO)cc3C2=O)C(=O)N1]

BH₃-THF (1M in THF, 40 kg, 3.2 wt, 1.04 eq) is added to a slurry of 2-(2,6-dioxopiperidin-3-yl)-3-oxoisoindoline-5-carbaldehyde (12.55 kg without assay correaction, 1 wt, 1.0 eq.) in DMAc (90 kg, 7.2 wt) at 20-30° C. (2 h addition time, mild exothermic and gas evolution). The resulting mixture is stirred at 20-30° C. until the reaction is complete. Adjust to 5-30° C. The reaction mixture is quenched by MeOH (18 kg, 1.4 wt) addition at 0-30° C. (2 h addition time, strong exothermic and gas evolution at the beginning), then stirred at 20-30° C. for 3 h. Concentration to ca. 4 vol in vacuo below 80° C. and dilution with EtOH (88 kg, 7.0 wt) at 20-30° C. within 3 h. After aging at 20-30° C. for 5 h, crude 3-(6-(hydroxymethyl)-1-oxoisoindolin-2-yl) piperidine-2,6-dione is isolated by filtration and washing with EtOAc (21 kg, 1.7 wt). The wet cake is slurried in EtOAc (50 kg, 4.0 wt) at 20-30° C. for 3 h. After filtration and EtOAc wash (20 kg, 1.6 wt), wet 3-(6-(hydroxymethyl)-1-oxoisoindolin-2-yl) piperidine-2,6-dione is obtained and dried at 45-55° C. for 44 h under reduced pressure to give purified 3-(6-(hydroxymethyl)-1-oxoisoindolin-2-yl) piperidine-2,6-dione as as pale gray solid (9.75 kg with 88.8% assay in 68% yield).

### Stage 3.

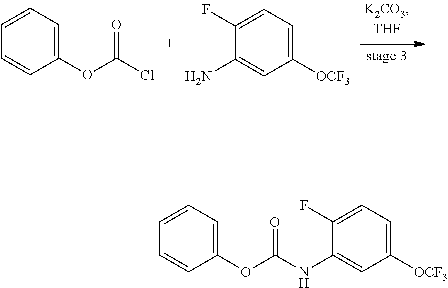 [SMILES: O=C(Cl)Oc1ccccc1 ; Nc1cc(OC(F)(F)F)ccc1F ; O=C(Nc1cc(OC(F)(F)F)ccc1F)Oc1ccccc1]

### Exemplary Preparation 1 of phenyl (2-fluoro-5-(trifluoromethoxy)phenyl)carbamate

To a suspension of 2-fluoro-5-(trifluoromethoxy) aniline (6.0 kg, 1 wt, 1.0 wt) and K₂CO₃ (5.5 kg, 0.92 wt, 1.3 eq) in THF (40 kg, 6.7 wt) is added phenyl carbonochloridate (4.75 kg, 0.8 wt, 1.0 eq) at 15-25° C. (1.5 h addition time, mild exothermic), rinsed with THF (13 kg, 2.2 wt). Complete conversion is achieved after stirring at 15-25° C., then the mixture is filtered and rinsed with THF (24 kg, 4.0 wt). The filtrate is concentrated under reduced pressure below 40° C. to 1-3 vol. THF (15.5 kg, 2.6 wt) is added and concentrated under reduced pressure below 40° C. to 1-3 vol to yield phenyl (2-fluoro-5-(trifluoromethoxy)phenyl)carbamate as concentrated solution in THF which is stored shortly and used as such (9.69 kg theory amount. The solution is concentrated to the estimated volume and transformed from reactor to reactor. The weight of solution is not available).

### Exemplary Preparation 2 of phenyl (2-fluoro-5-(trifluoromethoxy)phenyl)carbamate

To a suspension of 2-fluoro-5-(trifluoromethoxy) aniline (3.28 kg, 1 wt, 1.0 eq) and K₂CO₃ (3.0 kg, 0.9 wt, 1.3 eq) in THF (31.7 kg, 9.7 wt) is added phenyl carbonochloridate (2.66 kg, 0.8 wt, 1.0 eq) at 15-25° C. (ca. 2 h addition time, mild exothermic), rinsed with THF (1.4 kg, 0.4 wt). Complete conversion is achieved after stirring at 15-25° C. for 4 h, then the mixture is filtered and rinsed with THF (6 kg, 1.8 wt). The filtrate is concentrated under reduced pressure below 40° C. to 1-3 vol. THF (9 kg, 2.7 wt) is added and concentrated under reduced pressure below 40° C. to 1-3 vol to yield phenyl (2-fluoro-5-(trifluoromethoxy)phenyl)carbamate as concentrated solution in THF which is stored shortly and used as such (12.7 kg THF solution, 5.30 kg theory amount).

### Stage 4.

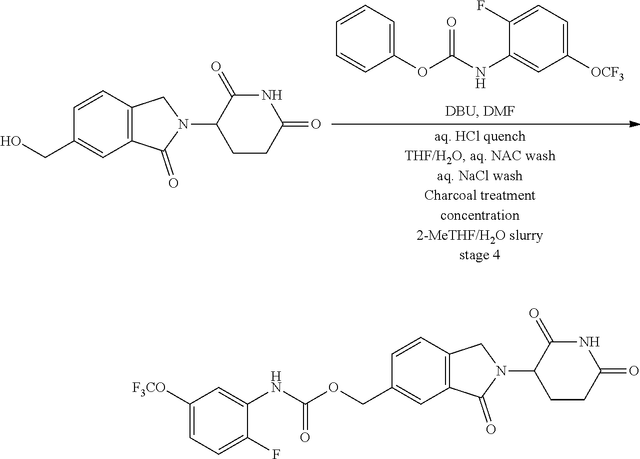 [SMILES: O=C1CCC(N2Cc3ccc(CO)cc3C2=O)C(=O)N1 ; O=C(Nc1cc(OC(F)(F)F)ccc1F)Oc1ccccc1 ; O=C1CCC(N2Cc3ccc(COC(=O)Nc4cc(OC(F)(F)F)ccc4F)cc3C2=O)C(=O)N1]

### Exemplary Preparation 1 of (2-(2,6-dioxopiperidin-3-yl)-3-oxoisoindolin-5-yl) methyl (2-fluoro-5-(trifluoromethoxy)phenyl)carbamate

Freshly prepared phenyl (2-fluoro-5-(trifluoromethoxy)phenyl)carbamate as THF solution (9.69 kg theory amount, 1.8 wt, 1.5 eq) from step 3 is added to a solution of 3-(6-(hydroxymethyl)-1-oxoisoindolin-2-yl) piperidine-2,6-dione (6.24 kg*88.8%=5.5 kg, 1.0 wt) and DBU (9.4 kg, 1.7 wt, 3.0 eq) in DMF (34 kg, 6.2 wt) at 10-20° C. (mild exothermic, ca. 2 h). DMF (11 kg, 2.0 wt) and THF (7 kg, 1.3 wt) is added to rinse the reactor and pipeline. Stirring at 10-20° C. is continued until the reaction is complete. The resulting mixture is dosed into an aqueous solution of 0.6M HCl (123 kg, 22.4 wt) at 15-25° C. in 1 h. After aging at 15-25° C. for 6 h, crude product (2-(2,6-dioxopiperidin-3-yl)-3-oxoisoindolin-5-yl) methyl (2-fluoro-5-(trifluoromethoxy)phenyl)carbamate is filtered and washed with purified water (11.6 kg, 2.1 wt). After intermediate drying at 15-25° C. for 7 h, the wet material (26.36 kg, 4.8 wt) is taken up in THF (72 kg, 13.1 wt) and the organic layer washed with aqueous 10 wt % N-acetyl cysteine/Na₂CO₃ solution (65 kg, 11.8 wt). After back-extracting the aqueous phase with THF (40 kg, 7.3 wt) at 20-30° C. for 1 h, combined organic phases are washed with 10 wt % N-acetyl cysteine/Na₂CO₃ (33 kg, 6.0 wt) and 25% w/w NaCl twice (29 kg, 5.3 wt and 28 kg, 5.1 wt. The interface is not obvious and judged by the weight of aqueous layer phase). The resulting organic layer is filtered (almost no solid, transferred to a new reactor and some black oil was observed on the previous reactor) and washed with 20:1 V/V THF/H₂O (22 kg, 4.0 wt). The organic layer is concentrated to 2-6 vol under vacuum below 40° C. 2-MeTHF (38 kg, 6.9 wt) and purified water (58 kg, 10.5 wt) are added and the suspension stirred at 20-30° C. for 2 h. After filtration and purified water (15 kg, 2.7 wt), 2-MeTHF wash (10 kg, 1.8 wt), drying at 35-45° C. for 6 h then 45-55° C. for 17 h, crude (2-(2,6-dioxopiperidin-3-yl)-3-oxoisoindolin-5-yl) methyl (2-fluoro-5-(trifluoromethoxy)phenyl)carbamate is obtained as pale yellow solid (8.34 kg, 84% yield).

(2-(2,6-Dioxopiperidin-3-yl)-3-oxoisoindolin-5-yl) methyl (2-fluoro-5-(trifluoromethoxy)phenyl)carbamate (8.23 kg, 1 wt) is dissolved in DMSO (41.4 kg, 5.0 wt) at 20-30° C. for 2 h, filter into the reactor then rinse the pipeline with DMSO (4.6 kg, 0.6 wt). A mixture of 2:1 V/V EtOH/water (24.2 kg, 3.0 wt) added at 20-30° C. in 2 h followed by seeding (0.05 kg, 0.006 wt). After aging at 20-30° C. for 3 h, 1:1 V/V EtOH/water (109 kg, 13.3 wt) was charged at 20-30° C. in 9 h. After aging at 20-30° C. for 4 h, suspension is filtered and rinsed with EtOH (21.0 kg, 2.6 wt). Slurry the wet cake with EtOH (60.0 kg, 7.3 wt) in filter dryer at 45-55° C. for 6 h, adjust to 20-30° C. in 5 h and aged at 20-30° C. for 2 h. Filtration and wash with EtOH (27.0 kg, 3.3 wt). Drying at 15-25° C. for 4 h provide (2-(2,6-dioxopiperidin-3-yl)-3-oxoisoindolin-5-yl) methyl (2-fluoro-5-(trifluoromethoxy)phenyl)carbamate with intermediate purity (7.59 kg, 0.93 wt). The solid is charged into DMSO (24 kg, 2.9 wt) and slurried at 45-55° C. for 7 h, adjust to 20-30° C. for 7 h, followed by aging at 20-30° C. for 3 h. EtOH (132 kg, 16.1 wt) is added in 9 h at 20-30° C. and the suspension further stirred at 20-30° C. for 4 h. After filtration and EtOH wash (22.5 kg, 2.7 wt), the wet cake is slurried with EtOH (61.0 kg, 7.4 wt) in filter dryer at 45-55° C. for 3 h, temperature adjusted to 20-30° C. in 4 h and aged at 20-30° C. for 1 h. After filtration in filter dryer and wash with EtOH (23.0 kg, 2.8 wt) followed by drying at 35-45° C. for 7 h under vacuum, (2-(2,6-dioxopiperidin-3-yl)-3-oxoisoindolin-5-yl) methyl (2-fluoro-5-(trifluoromethoxy)phenyl)carbamate is obtained as white solid (6.62 kg, 80% yield).

### Exemplary Preparation 2 of (2-(2,6-dioxopiperidin-3-yl)-3-oxoisoindolin-5-yl) methyl (2-fluoro-5-(trifluoromethoxy)phenyl)carbamate

Freshly prepared phenyl (2-fluoro-5-(trifluoromethoxy)phenyl)carbamate as THF solution (2.59 kg theory amount, 1.7 wt, 1.5 eq) from step 3 is added to a solution of 3-(6-(hydroxymethyl)-1-oxoisoindolin-2-yl) piperidine-2,6-dione (1.70 kg*88.8%=1.51 kg, 1.0 wt, 1.0 eq) and DBU (2.5 kg, 1.7 wt, 3.0 eq) in DMF (11.6 kg, 7.7 wt) at 10-20° C. (mild exothermic, ca. 1.5 h). DMF (0.6 kg, 0.4 wt) is added to rinse the reactor and pipeline. Stirring at 10-20° C. is continued for 3 h. The resulting mixture is quenched into an aqueous solution of 0.6M HCl (33.5 kg, 22.2 wt) at 15-25° C. in 3 h. After aging at 15-25° C. for 5 h, crude product (2-(2,6-dioxopiperidin-3-yl)-3-oxoisoindolin-5-yl) methyl (2-fluoro-5-(trifluoromethoxy)phenyl)carbamate is filtered and washed with process water (2.8 kg, 1.9 wt). The wet material (6.58 kg, 4.4 wt) is taken up in THF (48 kg, 32.8 wt) and the organic layer washed with aqueous 10 wt % N-acetyl cysteine/Na₂CO₃ solution (19 kg, 12.6 wt). After back-extracting the aqueous phase with THF (11 kg, 7.3 wt), combined organic phases are washed with 10 wt % N-acetyl cysteine/Na₂CO₃ (9 kg, 6.0 wt) and 25% w/w NaCl twice (8 kg*2, 5.3 wt*2). The resulting organic layer is filtered and washed with 20:1 V/V THF/H₂O (9 kg, 6.0 wt). The organic layer is transferred to the reactor and rinse with THF (0.5 kg, 1.0 wt). Recycle the THF solution through CUNO (ZetaCarbon, Grade: R55SP, Diameter: 8 inch, Area: 0.35 m², Actived carbon: 0.3 kg) at 35-45° C. for 6 h. The organic layer is transferred to the reactor and rinse with THF (4.3 kg, 4.0 wt), then concentrated to 2-6 vol under vacuum below 40° C. 2-MeTHF (11 kg, 7.3 wt) and purified water (17 kg, 11.3 wt) are added and the suspension stirred at 20-30° C. for 2 h. After filtration and 2-MeTHF wash (3 kg, 2.0 wt), drying at 45-55° C. for 16 h, crude (2-(2,6-dioxopiperidin-3-yl)-3-oxoisoindolin-5-yl) methyl (2-fluoro-5-(trifluoromethoxy)phenyl)carbamate is obtained as crude product (2.08 kg, pale yellow solid, 77% yield).

### Exemplary Preparation 3 of (2-(2,6-dioxopiperidin-3-yl)-3-oxoisoindolin-5-yl) methyl (2-fluoro-5-(trifluoromethoxy)phenyl)carbamate

Freshly prepared phenyl (2-fluoro-5-(trifluoromethoxy)phenyl)carbamate as THF solution (2.59 kg theory amount, 1.7 wt, 1.5 eq) from step 3 is added to a solution of 3-(6-(hydroxymethyl)-1-oxoisoindolin-2-yl) piperidine-2,6-dione (1.69 kg*88.8%=1.50 kg, 1.0 wt) and DBU (2.48 kg, 1.7 wt, 3.0 eq) in DMF (11.6 kg, 7.7 wt) at 10-20° C. (mild exothermic, ca. 2 h). DMF (0.6 kg, 0.4 wt) is added to rinse the reactor and pipeline. Stirring at 10-20° C. is continued for 3 h. The resulting mixture is quenched into an aqueous solution of 0.6M HCl (33.5 kg, 22.3 wt) at 15-25° C. in 2 h. After aging at 15-25° C. for 5 h, crude product (2-(2,6-dioxopiperidin-3-yl)-3-oxoisoindolin-5-yl) methyl (2-fluoro-5-(trifluoromethoxy)phenyl)carbamate is filtered and washed with process water (3 kg, 2.0 wt). The wet material (6.52 kg, 4.3 wt) is taken up in THF (49 kg, 32.7 wt) and the organic layer washed with aqueous 10 wt % N-acetyl cysteine/Na₂CO₃ solution (19 kg, 12.7 wt). After back-extracting the aqueous phase with THF (11 kg, 7.3 wt), combined organic phases are washed with 10 wt % N-acetyl cysteine/Na₂CO₃ (9 kg, 6.0 wt) and 25% w/w NaCl twice (8 kg*2, 5.3 wt*2). The resulting organic layer is filtered and washed with 20:1 V/V THF/H₂O (9 kg, 6.0 wt). The organic layer is transferred to the reactor and rinse with THF (1.5 kg, 1.0 wt). Recycle the THF solution through CUNO (ZetaCarbon, Grade: R55SP, Diameter: 8 inch, Area: 0.35 m², Actived carbon: 0.3 kg) at 35-45° C. for 6 h. The organic layer is transferred to the reactor and rinse with THF (6.0 kg, 4.0 wt), then concentrated to 2-6 vol under vacuum below 40° C. 2-MeTHF (11 kg, 7.3 wt) and purified water (17 kg, 11.3 wt) are added and the suspension stirred at 20-30° C. for 2 h. After filtration and 2-MeTHF wash (3 kg, 2.0 wt), drying at 45-55° C. for 16 h, crude (2-(2,6-dioxopiperidin-3-yl)-3-oxoisoindolin-5-yl) methyl (2-fluoro-5-(trifluoromethoxy)phenyl)carbamate is obtained as crude product (2.16 kg, pale yellow solid, 80% yield).

### Stage 5.

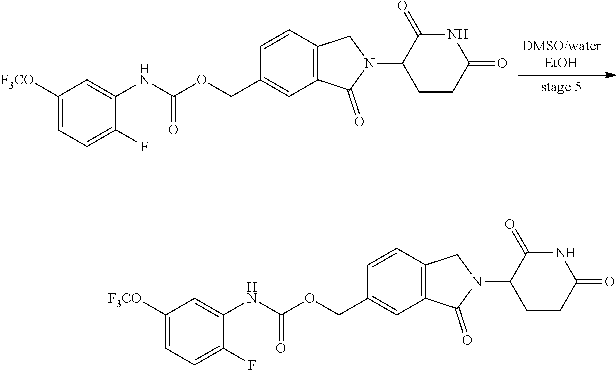 [SMILES: O=C1CCC(N2Cc3ccc(COC(=O)Nc4cc(OC(F)(F)F)ccc4F)cc3C2=O)C(=O)N1 ; O=C1CCC(N2Cc3ccc(COC(=O)Nc4cc(OC(F)(F)F)ccc4F)cc3C2=O)C(=O)N1]

### Exemplary Preparation 1 of (2-(2,6-dioxopiperidin-3-yl)-3-oxoisoindolin-5-yl) methyl (2-fluoro-5-(trifluoromethoxy)phenyl)carbamate

(2-(2,6-Dioxopiperidin-3-yl)-3-oxoisoindolin-5-yl) methyl (2-fluoro-5-(trifluoromethoxy)phenyl)carbamate (4.7 kg, 1 wt) is divided into 3 batches. Each batch (1.57 kg, 0.33 wt) is dissolved in THF/H₂O (V/V=20:1, ca. 86 kg, 18 wt) at 25-35° C., recycle through CUNO (ZetaCarbon, Grade: R55SP, Diameter: 8 inch, Area: 0.35 m², Actived carbon: 0.3 kg) for 6 h. The combined solution is concentrated to 2-4 vol under vacuum below 40° C. 2-MeTHF (34 kg, 7.2 wt) and purified water (50 kg, 10.6 wt) are added and the suspension stirred at 20-30° C. for 3 h. After filtration and 2-MeTHF wash (6 kg, 1.3 wt), (2-(2,6-dioxopiperidin-3-yl)-3-oxoisoindolin-5-yl) methyl (2-fluoro-5-(trifluoromethoxy)phenyl)carbamate is obtained after drying at 20-40° C. for 8 h (4.57 kg, 0.97 wt). The solid is charged into DMSO (12 kg, 2.6 wt) and slurried at 45-55° C. for 7 h, adjust to 20-30° C. for 7 h. EtOH (70 kg, 14.9 wt) is added in 7 h at 20-30° C. and the suspension further stirred at 20-30° C. for 3 h. Filtration and wash with EtOH (15 kg, 3.2 wt), then drying at 45-55° C. for 14 h, (2-(2,6-dioxopiperidin-3-yl)-3-oxoisoindolin-5-yl) methyl (2-fluoro-5-(trifluoromethoxy)phenyl)carbamate (3.88 kg, 0.83 wt) is obtained as crude product. The solid is charged into DMSO (12 kg, 2.6 wt) and slurried at 45-55° C. for 17 h, adjust to 20-30° C. for 7 h. EtOH (70 kg, 15 wt) is added in 7 h at 20-30° C. and the suspension further stirred at 20-30° C. for 3 h. Filtration and wash with EtOH (10 kg, 2.1 wt), then drying at 45-55° C. for 13 h, (2-(2,6-dioxopiperidin-3-yl)-3-oxoisoindolin-5-yl) methyl (2-fluoro-5-(trifluoromethoxy)phenyl)carbamate is obtained as white solid (3.06 kg, 65% yield)

### Exemplary Preparation 2 of (2-(2,6-dioxopiperidin-3-yl)-3-oxoisoindolin-5-yl) methyl (2-fluoro-5-(trifluoromethoxy)phenyl)carbamate

(2-(2,6-Dioxopiperidin-3-yl)-3-oxoisoindolin-5-yl) methyl (2-fluoro-5-(trifluoromethoxy)phenyl)carbamate (4.24 kg, 1 wt) is dissolved in DMSO (21 kg, 5.0 wt) at 20-30° C. for ca.2 h, filter into the reactor then rinse the pipeline with DMSO (2 kg, 0.6 wt). A mixture of 2:1 V/V EtOH/water (11 kg, 3.0 wt) added at 20-30° C. in 2 h followed by seeding (0.02 kg, 0.006 wt). After aging at 20-30° C. for 1.5 h, 1:1 V/V EtOH/water (38 kg, 13.3 wt) was charged at 20-30° C. in 7.5 h. After aging at 20-30° C. for 4 h, suspension is filtered and rinsed with EtOH (8 kg, 2.6 wt). The solid is charged into DMSO (11.7 kg, 2.9 wt) and slurried at 45-55° C. for 8 h, adjust to 20-30° C. for 7 h. EtOH (61.2 kg, 16.1 wt) is added in 8 h and the suspension is further stirred at 20-30° C. for 3 h. After filtration and EtOH wash (8 kg, 2.7 wt). The wet cake is charged into DMSO (10.7 kg, 2.9 wt) and slurried at 45-55° C. for 8 h, adjust to 20-30° C. for 7 h. EtOH (57.9 kg, 16.1 wt) is added in 7 h and the suspension is further stirred at 20-30° C. for 3 h. After filtration and EtOH wash (8 kg, 2.7 wt) followed by drying at 45-55° C. for 20 h under vacuum, (2-(2,6-dioxopiperidin-3-yl)-3-oxoisoindolin-5-yl) methyl (2-fluoro-5-(trifluoromethoxy)phenyl)carbamate is obtained as white solid (3.43 kg, 80% yield).

### Example 3: Compound Activity by Fluorescent Polarization Assay

Compound activity was monitored in a fluorescence polarization (FP) homogeneous assay using 1-[5-({2-[2-(2-{[2-(2,6-dioxopiperidin-3-yl)-1,3-dioxo-2,3-dihydro-1H-isoindol-4-yl]oxy}acetamido) ethoxy]ethyl}carbamoyl) pentyl]-3,3-dimethyl-2-[(1E,3E)-5-[(2E)-1,3,3-trimethyl-5-sulfo-2,3-dihydro-1H-indol-2-ylidene]penta-1,3-dien-1-yl]-3H-indol-1-ium-5-sulfonate as a fluorescent probe. Unless otherwise stated, all reagents were purchased from Sigma Aldrich. Enzymatic reactions were conducted in Perkin-Elmer Black 384 well ProxiPlate Plus (catalogue no. 6008269) in 10 μL total volume. Full length wild-type cereblon CRBN (80.0 nM, 10 μL) was incubated in assay buffer containing 20 mM HEPES (pH 8.0), 150 NaCl, 0.5 mM TCEP and 0.05% Tween 20 in the presence or absence of compound (300 nL). Inhibitors were stored as 10 mM DMSO stocks in an inert environment (low humidity, dark, low oxygen, room temperature) using the Storage Pod System. Compounds and DMSO were dispensed using the Echo E5XX (Labcyte Inc. USA) to give concentrations from 300 to 0.937 or 3000 to 9.3 nM in a 12 data point curve. Mutant YWAA CRBN (80.0 nM, 10 μL) which does not interact with the fluorescent probe was used as a negative control for the assay. Following incubation at room temperature for 30 min, the assay was initiated by dispensing the probe to a final concentration of 5 nM (2.5 nL of a 20 μM stock) using the Echo E5XX. FP was measured after a period of 12 hours using a Pherastar plate reader (BMG Labtech, Germany) exciting at 590 nm and measuring the amount of parallel and perpendicular light at 675 nm. The FP signal was subsequently normalized to the no-compound control (i.e., DMSO). Analysis and IC50 values were derived using Dotmatics (Dotmatics UK) software.

Table 1 assigns each compound a code indicating the ability for cereblon binding by measn of their IC50 values. According to the code, B represents an IC50 value >500 nM and ≤1100 nM.

TABLE 1
IC50 values determined in the fluorescence polarization
assay indicating the cereblon binding

| Compound | rFP IC50 [nM] |
|---|---|
| 145 | B |

### Example 4: Compound Binding by Immunofluorescence Assay

The representative compounds were tested in an immunofluorescence assay for their activity to bind to degrade GSPT1. CAL-51 cells were purchased from DSMZ (cat. Number ACC302), sub-cultured in 90% Dulbecco's MEM (4.5 g/L glucose, Gibco 11965)+10% heat inactivated FBS (BioConcept, 2-01F136I) and incubated at 37° C., 5% CO₂. For the assay, imaging microtiterplate Cell Carrier 96 Ultra (Perkin Elmer 6055302) were pre-coated with Fibronectin (Sigma F085, 30 μl at 0.2 μg/ml) in PBS (100 μl, Gibco 14190) for 45 min at room temperature, rinsed with PBS and CAL-51 cells (30K cells/well) were plated and let to adhere overnight. Cells were treated with compounds typically using a serial dilution ranging from 30 μM to 0.1 nM for 6 hours. Compounds were stored at 10 mM DMSO stocks. Vehicle (DMSO), positive (CC-885, 10 μM) and rescue controls (positive control plus 0.2 μM bortezomib) were also included atthis stage. Cells were subsequently rinsed with PBS and fixed in 10% Formalin solution (50 μl, Sigma HT5011)) for 20 mins at room temperature. Following three consecutive PBS washes (100 μl), cells were permeabilized in 0.1% Triton X-100 in PBS (Sigma 93443, 50 μl) for 15 mins at room temperature. Following three further PBS washes, 50 μl blocking buffer (1% BSA, Sigma A4503, in PBS) was added for 45 min for signal-to-noise reduction.

Primary antibody (human GSPT1, Sigma HPA052488) was diluted in blocking buffer (dil.1/300, 35 μl/well) and incubated with the cells overnight at 4° C. After three PBS washes, Alexa-fluor 488 coupled secondary antibodies (Invitrogen, A32731, dil.1/1000), Alexa-fluor 647-Phalloïdin (Invitrogen, A22287, dil. 1/200) and DAPI (Thermo, #62248, dil.1/1000) were diluted in blocking buffer and incubated with the samples for 2 hours at room temperature. After three final PBS washes, samples were conserved in 100 μl PBS in the dark, until measurement. Image acquisition was performed on the Operetta High-Content Imager (Perkin-Elmer). Fluorescence intensity of Alexa-Fluor 488 (GSPT1), Alexa-Fluor 647 (Actin) and DAPI (Nucleus) were measured. For the determination of GSPT1 DC₅₀ values, a custom algorithm implemented in the PerkinElmer image analysis software Harmony-Acapella® was developed. After user-defined setting of adjustment parameters, the analysis was run identically without human intervention for all image fields. DAPI staining of the nuclei was used to determine the location of cells using standard nuclei detection modules. Segmentation artifacts were removed by threshold-based filters for area, roundness and intensity. The outline of the cells was determined analogously from the sum of the normalized, smoothed DAPI and Actin channel, starting from each nucleus.

The Alexa-Fluor 488 (GSPT1) signal intensity in each cell was finally measured, in order to obtain a Mean intensity per cell. GSPT1 degradation (DC₅₀) was calculated after normalization to controls and data import in CDD vault Database, using non-linear regression.

Table 2 assigns each compound a code indicating the ability for GSPT1 degradation. According to the code, B represents a DC50 value >30 nM and ≤300 nM.

TABLE 2
Activity for GSPT1 degradation

| | |
|---|---|
| Compound 145 | B |

### Example 5: Methods of Preparing and Characterization of Crystalline Form I

Crystalline Form I was prepared by the protocol described in Example 2 and Example 2A. The XRPD results were generated under the diffraction method parameters shown in Table 3.

TABLE 1
XRPD method parameters

| Method Name | Method 1 | Sensing Pattern | Step-measuring |
|---|---|---|---|
| X-ray Genrator | Cu Kα (λ = 1.54184 Å) | Tube Voltage(kV) | 30 |
| Tube Current(mA) | 10 | Divergence Slit(mm) | 0.6 |
| Primary Soller Slit(°) | 2.5 | Secondary Silt(°) | 2.5 |
| Detector Slit(°) | 5.827 | Antiscattering Silt(mm) | 0 |
| Scan axis | θs-θd | Step size(deg) | 0.02 |
| Time/step (S) | 0.2 | Scanning Scope(deg) | 3-40 |

XRPD patterns of crystalline Form I is shown in FIG. 1. Table 4 list certain XRPD characteristic peaks for crystalline Form I.

TABLE 4
Exemplary XRPD patterns of crystalline Form I

| 2θ (deg.) | d Value (Å) | Relative Intensity |
|---|---|---|
| 11.35 | 7.70 | 47.3% |
| 11.96 | 7.39 | 35.5% |
| 12.90 | 6.86 | 24.4% |
| 16.36 | 5.41 | 15.8% |
| 18.83 | 4.71 | 70.7% |
| 19.72 | 4.50 | 47.0% |
| 22.86 | 3.89 | 48.6% |
| 23.70 | 3.75 | 100% |
| 25.94 | 3.43 | 24.9% |
| 27.31 | 3.26 | 17.9% |

The DSC curves were generated by DSC214 of NETZSCH Group or DSC 3 of Mettler. The parameters of DSC method are presented as Table 5.

TABLE 5
DSC method parameters

| Crucible | High pressure, steel |
|---|---|
| Starting temperature (° C. ) | 30 |
| Ending temperature (° C. ) | 350 |
| Heating rate (° C./min) | 10 |

DSC curves of crystalline Form I is shown in FIG. 2.

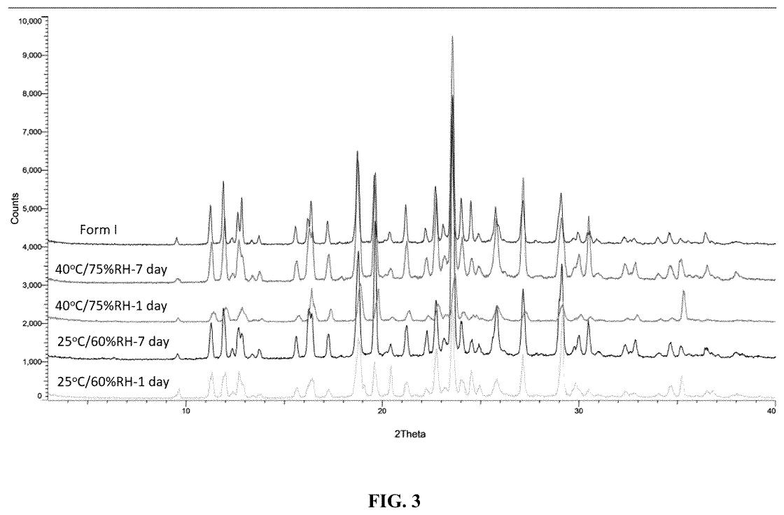

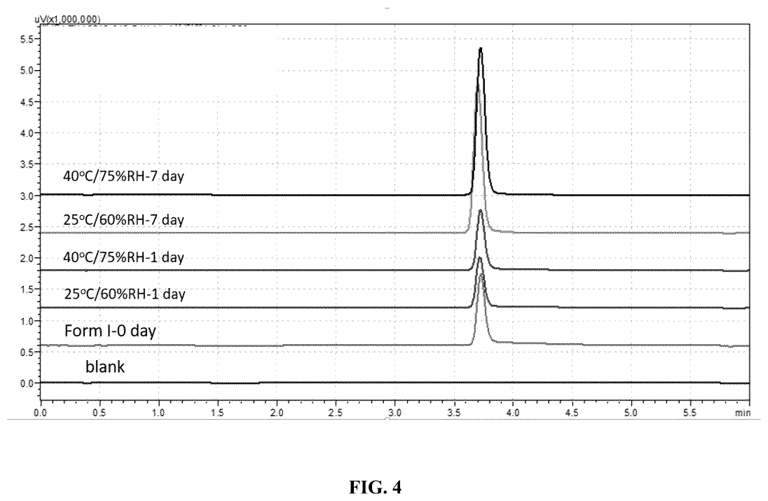

Solid stability was evaluated at 40° C./75RH and 25° C./60% RH for 7 days. The results were summarized in Table 6, FIG. 3 and FIG. 4. Table 6 shows the purity of crystalline Form I at day 1 and day 7 in 40° C./75% RH and 25° C./60% RH. FIG. 3 shows the XRPD pattern of crystalline Form I at day 1 and day 7 in 40° C./75% RH and 25° C./60% RH. FIG. 4 shows the HPLC data of crystalline Form I at day 1 and day 7 in 40° C./75% RH and 25° C./60% RH. As shown in Table 6, FIG. 3 and FIG. 4, no obvious degradation nor form change was observed under testing conditions. Crystalline Form I was physically and chemically stable at 40° C./75% RH and 25° C./60% RH for 7 days.

TABLE 6
Stability results for crystalline Form I

| | Purity- | 25° C./60% RH | | | | 40° C./75% RH | | | |
|---|---|---|---|---|---|---|---|---|---|
| Sample | T0 | Purity | | XRPD | | Purity | | XRPD | |
| Form I | 100% | 1 day | 7 day | 1 day | 7 day | 1 day | 7 day | 1 day | 7 day |
| | | 100% | 100% | Form I | Form I | 100% | 99.95% | Form I | Form I |

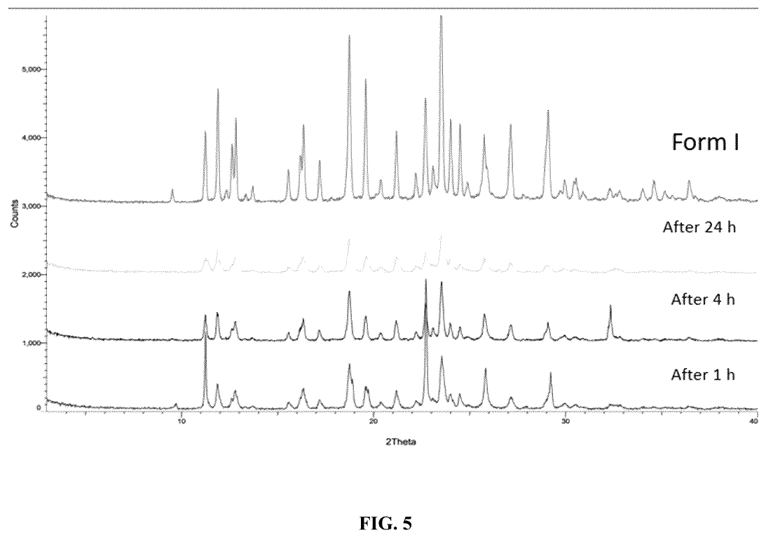

Solubility of crystalline Form I was evaluated by weighing about 10 mg of crystalline Form I into 2 mL of media, and then stirring the suspension at 37° C. for 24 hours. At 1, 4 and 24 hours, the suspensions were filtered, and the filtrates were analyzed by HPLC and pH. The solubility of the starting material (Form I) in media is summarized in Table 7. As shown in FIG. 5, the crystal form of remained solid in all media were unchanged.

TABLE 7
Solubility results for crystalline Form I

| | Solubility at 37° C. (mg/mL) | | | | XRPD(24 |
|---|---|---|---|---|---|
| NO. | 1 h | 4 h | 24 h | pH(24 h) | h) |
| H₂O-1 | <0.001 | <0.001 | <0.001 | 6.35 | Form I |
| H₂O-2 | <0.001 | <0.001 | <0.001 | 6.61 | Form I |
| H₂O-3 | <0.001 | <0.001 | <0.001 | 6.41 | Form I |

Form I is an anhydrate with high crystallinity, and nonhydroscopic. Form I has good physical and chemical stability when placed in 25° C. 60% RH and 40° C. 75% RH for 7 days. The solubility of Form I in water was less than 0.001 mg/mL.

### Example 6: Methods of Preparing and Characterization of Crystalline Form II

Crystalline Form II was prepared by evaporation crystallization in a 96-well plate. Specifically, Compound 145 was placed in a solution of solvent and then subjected to slow evaporation at RT (25-28° C.) to induce precipitation. The solutions that formed crystalline Form II are: acetone, methyl ethyl ketone (MEK), methyl isobutyl ketone (MIBK), ethyl acetate (EA), and heptanone. The XRPD results were generated. The diffraction method parameters are shown in Table 3 above. An XRPD pattern of crystalline Form II is shown in FIG. 6.

### Example 7: Methods of Preparing and Characterization of Crystalline Form III

Crystalline Form III was prepared by evaporation crystallization in 1,4-dioxane. About 200 mg of Compound 145 was combined with 20 mL of 1,4-dioxane. The mixture was stirred at 25-28° C. for 30 minutes. After filtration, the filtrate was slowly evaporated at 25-28° C.

The XRPD results were generated. The diffraction method parameters are shown in Table 3 above. An XRPD pattern of crystalline Form III is shown in FIG. 7. The XRPD results showed that Form III had poor crystallinity.

The DSC curves were generated by DSC214 of NETZSCH Group or DSC 3 of Mettler. The parameters of DSC method are presented as Table 9 above. DSC curves of crystalline Form I is shown in FIG. 8. The DSC results show that Form III may be transformed into Form I during DSC heating.

1. A process for preparing a compound having the structure: 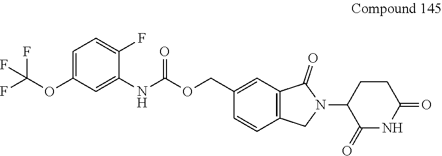 [SMILES: O=C1CCC(N2Cc3ccc(COC(=O)Nc4cc(OC(F)(F)F)ccc4F)cc3C2=O)C(=O)N1]
the process comprising:
providing Compound 1 having the structure: 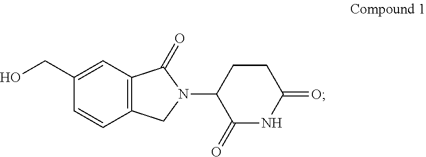 [SMILES: O=C1CCC(N2Cc3ccc(CO)cc3C2=O)C(=O)N1]
providing Compound 1 having the structure: 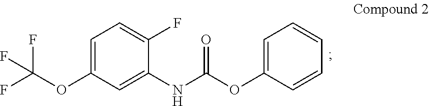 [SMILES: O=C(Nc1cc(OC(F)(F)F)ccc1F)Oc1ccccc1]
and reacting Compound 1 and Compound 2 in the presence of an organic base.

2. The process of claim 1, wherein the organic base is an amine base.

3. (canceled)

4. The process of claim 1, comprising providing Compound 3 having the structure: 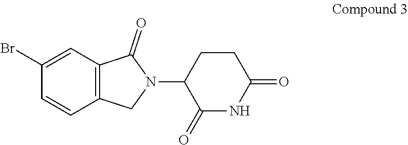 [SMILES: O=C1CCC(N2Cc3ccc(Br)cc3C2=O)C(=O)N1]
and reacting Compound 3 with carbon monoxide in the presence of a catalyst to produce Compound 4 having the structure:

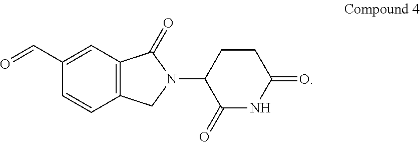 [SMILES: O=Cc1ccc2c(c1)C(=O)N(C1CCC(=O)NC1=O)C2]

5. (canceled)

6. The process of claim 4, comprising reacting Compound 3 and carbon monoxide in the presence of a base.

7. The process of claim 4, comprising reacting Compound 4 with a borane reactant to produce Compound 1.

8. The process of claim 1, comprising providing Compound 5 having the structure: 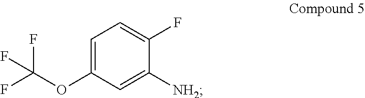 [SMILES: Nc1cc(OC(F)(F)F)ccc1F]
providing Compound 6 having the structure: 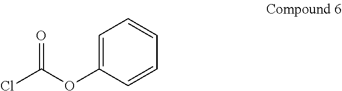
and reacting Compound 5 and Compound 6 in the presence of a base to produce Compound 2.

9. The process of claim 1, comprising purifying Compound 145, wherein purifying comprises (i) dissolving Compound 145 in a first solvent to produce a solution and (ii) adding a second solvent to the solution to produce purified Compound 145.

10. The process of claim 1, comprising crystallizing Compound 145 from ethanol, water, dimethyl sulfoxide, or a combination thereof.

11. (canceled)

12. A crystalline form of a compound having the structure: 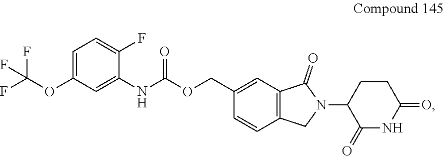 [SMILES: O=C1CCC(N2Cc3ccc(COC(=O)Nc4cc(OC(F)(F)F)ccc4F)cc3C2=O)C(=O)N1]
obtainable from the process according to claim 1.

13. A pharmaceutical composition comprising a crystalline form of a compound having the structure: 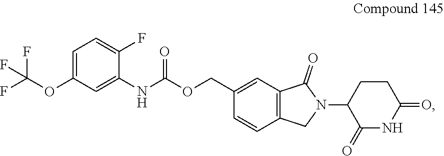 [SMILES: O=C1CCC(N2Cc3ccc(COC(=O)Nc4cc(OC(F)(F)F)ccc4F)cc3C2=O)C(=O)N1]
wherein the crystalline form is obtainable from the process according to claim 1.

14. A crystalline form of a compound having the structure: 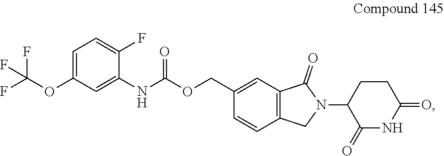 [SMILES: O=C1CCC(N2Cc3ccc(COC(=O)Nc4cc(OC(F)(F)F)ccc4F)cc3C2=O)C(=O)N1]
wherein the crystalline form is characterized as having an X-ray powder diffraction pattern as substantially shown in FIG. 1.

15. A crystalline form of a compound having the structure: 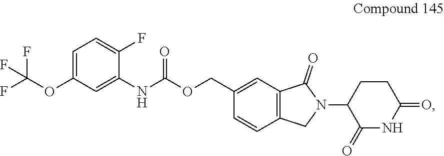 [SMILES: O=C1CCC(N2Cc3ccc(COC(=O)Nc4cc(OC(F)(F)F)ccc4F)cc3C2=O)C(=O)N1]
wherein the crystalline form is characterized by a powder X-ray diffraction pattern having characteristic peaks in degrees 2θ at 18.8°±0.2°, 22.9°±0.2°, and 23.7°±0.2°, wherein the X-ray diffraction pattern was obtained using Cu Kα radiation.

16. The crystalline form of claim 13, wherein the crystalline form is characterized by a powder X-ray diffraction pattern having characteristic peaks in degrees 2θ at 11.4°±0.2°, 18.8°±0.2°, 19.7°±0.2°, 22.9°±0.2°, and 23.7°±0.2°.

17. The crystalline form of claim 14, wherein the crystalline form is characterized by a powder X-ray diffraction pattern having characteristic peaks in degrees 2θ at 11.4°±0.2°, 12.0°±0.2°, 12.9°±0.2°, 16.4°±0.2°, 18.8°±0.2°, 19.7°±0.2°, 22.9°±0.2°, 23.7°±0.2°, 25.9°±0.2°, and 27.3°±0.2°.

18. The crystalline form of claim 12,
wherein the crystalline form is characterized as having an X-ray powder diffraction pattern as substantially shown in FIG. 6.

19. The crystalline form of claim 12,
wherein the crystalline form is characterized as having an X-ray powder diffraction pattern as substantially shown in FIG. 7.

20. (canceled)

21. The crystalline form of claim 12,
wherein the crystalline form maintains at least about 99 wt % of the compound upon storage under condition of 25° C. at 60% relative humidity for at least 7 days.

22. (canceled)

23. The pharmaceutical composition of claim 13, further comprising a pharmaceutically acceptable excipient.

24. (canceled)

25. (canceled)

26. A method of treating a cancer in a patient in need thereof, comprising administering to the patient the crystalline form of claim 12.

27. A method of treating a cancer in a patient in need thereof, comprising administering to the patient the pharmaceutical composition of claim 13.
